(19) United States
(12) Patent Application Publication
(10) Pub. No.: US 2002/0128215 A1
(43) Pub. Date: Sep. 12, 2002

(54) Novel sequence variants of the human N-acetyltransferase -2 (NAT -2) gene and use thereof

(76) Inventors: Hans-Ulrich Thomann, Lexington, MA (US); Kristen Wall, Hingham, MA (US); Michael Fitzgerald, Waltham, MA (US)

Correspondence Address: Nina L. Pearlmutter, Esq., Genome Therapeutics Corporation, 100 Beaver Street, Waltham, MA, 02421-4799, US

(21) Appl. No.: 09/776,407

(22) Filed: Feb. 2, 2001

Related U.S. Application Data
(60) Provisional application No. 60/179,876, filed on Feb. 2, 2000.

(51) Int. Cl. A61K 48/00; C12Q 1/68; C07H 21/04; C12N 9/00; C12N 5/06

(52) U.S. Cl. 514/44; 435/183; 435/325; 435/320.1; 536/23.2; 435/6

(57) ABSTRACT

This invention relates to novel polymorphisms of the NAT-2 gene which can be involved in drug metabolism and various disorders.

### RELATED APPLICATIONS

This application claims the benefit of U.S. Provisional Application No. 60/179,876, filed Feb. 2, 2000, the contents of which are incorporated in their entirety.

### FIELD

This invention relates to the field of molecular biology and genomics. The invention also relates to pharmacogenomics. The invention provides polymorphisms of the NAT-2 gene and the methods of using them in diagnostics and therapeutics.

### BACKGROUND

Many genetic variations are correlated with race and other genetically-related populations. Pharmacogenetic studies are used to identify the role of genetically-controlled variations in the response to drugs and other foreign compounds and provide a prognosis for a patient from a given population including a determination of the most effective drug and the drug dosage for a particular disorder or disease.

N-acetyltransferase 2 (NAT-2) is an enzyme that has been involved in several recent pharmacogenetic studies. It is an important enzyme because N-acetylation by hepatic arylamine N-acetyltransferase 2 (NAT-2) is a major route in the metabolism and detoxification of numerous drugs and foreign chemicals. Various polymorphisms of NAT-2 have been identified by others. The phenotypes resulting from these single nucleotide polymorphisms (SNPs) have been placed into two categories, slow-acetylators and rapid acetyaltors, depending on the activity of the NAT-2 enzyme. The phenotype is determined by the rate or degree of acetylation in liver of amine containing compounds. Those individuals in whom acetylation proceeds slowly are called slow acetylators and those in whom acetylation proceeds rapidly are rapid acetylators.

Weber et al. report that more than 50 percent of individuals in a Caucasian population were identified as slow acetylators (Pharmacol. Rev., 37, 25-79 (1985)). Slow acetylators demonstrated impaired metabolism of many therapeutic drugs including the anti-tuberculosis drug isoniazid, antidepressant phenylzine, antihypertensives hydrazine, the chemotherapeutic agents dapsone and amonafide, antiarrhythmic procainamide, sulfamethazine and other sulfonamides. (Weber, The Acetylator Genes and Drug Response, Oxford University Press, N.Y. (1987)). Adverse therapeutic effects of the acetylator phenotype include peripberal neuropathy and hepatitis; however, the N-acetylation of some drugs is beneficial to the individuals and reduces the drug's toxicity. Therefore understanding, an individuals genotype and resulting phenotype will assist physicians in designing a drug regimen which balances efficacy and toxicity.

NAT-2 also participates in activation pathways of environmental pollutants which have mutagenic-carcinogenic potential including, 2-aminofluorene, 4-aminobiphenyl, benzidine, beta-naphthylamine, and certain heterocyclic arylamines present in protein pyrolysates (see for example, Kato, CRC Crit. Rev. Toxicol., 16: 307-348 (1986); Weber, 1987, supra; Hein, Biochim Biophys Acta, 948:37-66 (1988)). Further, there has also been clinical evidence associating an acetylator phenotype with spontaneous or drug induced diseases such as bladder cancer (Evans, J. Med. Genet., 21:23-253 (1984)), colon cancer, prostate cancer, urothelial transitional cell carcinoma, Gilbert's disease (Platzer et al., Eur. J. Clin. Invest. 8: 219-223 (1978)), leprosy (Ellard et al., Nature, 239:159-160(1972)) and others (Evans, Pharmac. Ther. 42:157-234(1989)). NAT-2's participation in the detoxification has also been associated with chemically-induced disorders such as neoplasia (Vatsis et al., Pharmacogenetics, 5: 1-17 (1995)), and some activities including eating red meat and smoking in combination with NAT-2 phenotype have been shown to be associated with carcinogenesis. (Potter et al., Cancer Epidem. Biomarkers & Prev., 8: 69-75 (1999); and Liu et al., Canc. Letters, 133:115-123 (1998)).

Cascorbi et al. describe seven point mutations within the coding region of NAT-2. (Pharmogenetics, 9: 123-127 (1999)). Five of these mutations produce an amino acid change of which four produce the slow acetylator phenotype; however, some slow acetylator phenotypes have not been correlated with any known SNPS. (Hein et al., Hum.Mol. Genet., 3:729-734, (1994)). Thus there is a need for identifying additional mutations of the NAT-2 gene.

### BRIEF DESCRIPTION OF FIGURES

FIG. 1 depicts the N-acetyltransferase-2 gene regions amplified by oligonucleotide primers.

FIGS. 2A-2B depict the wild type NAT-2 gene (SEQ ID NO:1) These figures contain the nucleotide sequence of the wild type and the amino acid sequence (SEQ ID NO:2) starting at the “ATG” site, which is boxed. The base positions of the seven SNPs discovered are underlined in the figures and correspond to the base substitutions listed in Table 2. In addition, the amino acid changes are underlined.

### DEFINITIONS

“NAT-2 drug” refers to a compound that interacts with the NAT-2 gene. Preferably, a NAT-2 drug is metabolized by NAT-2 expressed product. Examples include, but are not limited to, amonafides, isoniazids, phenylzines, hydrazines, dapsones, procainamides, sulfamethazines and other sulfonamides.

“NAT-2 disorders” refer to disorders associated with the NAT-2 gene. Examples include, but are not limited to, bladder cancer, colon cancer, prostate cancer, Gilbert's disease, and leprosy.

“Amplification of nucleic acids” refers to methods such as polymerase chain reaction (PCR), ligation amplification (or ligase chain reaction, LCR) and amplification methods based on the use of Q-beta replicase. These methods are well known in the art and described, for example, in U.S. Pat. Nos. 4,683,195 and 4,683,202. Reagents and hardware for conducting PCR are commercially available. Primers useful for amplifying sequences from a specific chromosomal region are preferably complementary to, and hybridize specifically to sequences in a specific chromosomal region or in regions that flank a target region therein. The sequences generated by amplification may be sequenced directly. Alternatively, the amplified sequence(s) may be cloned prior to sequence analysis.

“Antibodies” refers to polyclonal and/or monoclonal antibodies and fragments thereof, and immunologic binding equivalents thereof, that can bind to proteins and polypeptides, and fragments thereof. The term antibody is used both to refer to a homogeneous molecular entity, or a mixture such as a serum product made up of a plurality of different molecular entities. Proteins can be prepared synthetically in a protein synthesizer and coupled to a carrier molecule and injected over several months into rabbits. Rabbit sera is tested for immunoreactivity to the protein, polypeptide, or fragment. Monoclonal antibodies can be made by injecting mice with the proteins, polypeptides, or fragments thereof. Monoclonal antibodies will be screened by ELISA and tested for specific immunoreactivity with NAT-2 protein or fragments thereof. Harlow et al, Antibodies: A Laboratory Manual, Cold Spring Harbor Laboratory, Cold Spring Harbor, N.Y. (1988). These antibodies will be useful in assays as well as pharmaceuticals. Antibody fragments can include Fa, F(ab′)₂, and Fv, which are capable of binding the epitopic determinant.

“cDNA” refers to complementary or copy DNA produced from an RNA template by the action of RNA-dependent DNA polymerase (reverse transcriptase). Thus, a “CDNA clone” means a duplex DNA sequence complementary to an RNA molecule of interest, carried in a cloning vector or PCR amplified.

“Cloning” refers to the use of in vitro recombination techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.

“cDNA library” refers to a collection of recombinant DNA molecules containing cDNA inserts which together comprise the entire genome of an organism. Such a cDNA library can be prepared by methods known to one skilled in the art and described by, for example, Cowell and Austin, “cDNA Library Protocols,” Methods in Molecular Biology (1997). Generally, RNA is first isolated from the cells of an organism from whose genome it is desired to clone a particular gene.

“Cloning vehicle” refers to a plasmid or phage DNA or other DNA sequence which is able to replicate in a host cell. The cloning vehicle is characterized by one or more endonuclease recognition sites at which such DNA sequences may be cut in a determinable fashion without loss of an essential biological function of the DNA, which may contain a marker suitable for use in the identification of transformed cells.

“Expression control sequence” refers to a sequence of nucleotides that control or regulate expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Expression control sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.

“Expression vehicle” refers to a vehicle or vector similar to a cloning vehicle but which is capable of expressing a gene which has been cloned into it, after transformation into a host. The cloned gene is usually placed under the control of (i. e., operably linked to) an expression control sequence.

“Gene” refers to a DNA sequence that encodes through its template or messenger RNA a sequence of amino acids characteristic of a specific peptide. The term “gene” includes intervening, non-coding regions, as well as regulatory regions, and can include 5′ and 3′ ends.

The gene sequences of the present invention can be derived from a variety of sources including DNA, cDNA, synthetic DNA, synthetic RNA or combinations thereof. Such sequences may comprise genomic DNA which may or may not include naturally-occurring introns. Moreover, such genomic DNA may be obtained in association with promoter regions or poly (A) sequences. The sequences, genomic DNA or cDNA can be obtained in any of several ways. Genomic DNA can be extracted and purified from suitable cells by means well known in the art. Alternatively, mRNA can be isolated from a cell and used to produce cDNA by reverse transcription or other means.

“Oligonucleotide” refers to a single stranded nucleic acid ranging in length from 2 to 60 bases. Oligonucleotides are often synthetic but can also be produced from naturally occurring polynucleotides. A probe is an oligonucloetide capable of binding to a target nucleic acid of complementary sequence through one or more types of chemical bonds,usually through complementary pairing via hydrogen bond formation. Oligonucleotides probes are often 5 to 60 bases and in specific embodiments may be between 10 and 40, or 15 and 30 bases long. An oligonucleotide probe may include natural (i.e. A, G, C or T) or modified bases (7-deazaguanosine, inosine, etc.). In addition, the bases may be joined by a linkage other than a phosphodiester bond, such as a phosphoramidite linkage or a phosphorothioate linkage, or they may be a peptide nucleic acids in which the constituent bases are joined by peptide bonds rather than by phosphodiester bonds, so long as it does not interfere with hybridization.

“Pharmacogenomics” or “pharmacogenetics” is the approach whereby a particular group of pharmaceutical agents are chosen to treat or diagnose disorders of an individual and/or class of individuals based on the polymorphisms of that individual or class. Pharmacogenomics or pharmacogenetics can also be used in the pharmaceutical research to assist in the drug selection process.

“Polymorphism” refers to the occurrence of two or more genetically or airtifically determined alternative sequences or alleles in a population.

As used herein, the term “primer” refers to a single-stranded oligonucleotide which acts as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not be perfectly complementary to the exact sequence of the template, but should be sufficiently complementary to hybridize with it. The term “primer site” refers to the sequence of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

“Reference sequence” is the nucleotide sequence of the NAT-2 gene (SEQ ID NO:1) and the corresponding amino acid sequence of the NAT-2 protein (SEQ ID NO:2) as described by Blum et al. (DNA and Cell Bio, 9:192-203(1990)). Genbank submission (AC number X14672).

“Single nucleotide polymorphism” or “SNP” occurs at a polymorphic site occupied by a single nucleotide which is the site of variation between allelic sequences.

“Host” includes prokaryotes and eukaryotes, such as bacteria, yeast and filamentous fungi, as well as plant and animal cells. The term includes an organism or cell that is the recipient of a replicable expression vehicle.

“Operator” refers to a DNA sequence capable of interacting with the specific repressor, thereby controlling the transcription of adjacent gene(s).

“Operably linked” means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

“Promoter” refers to a DNA sequence that can be recognized by an RNA polymerase. The presence of such a sequence permits the RNA polymerase to bind and initiate transcription of operably linked gene sequences.

“Promoter region” is intended to include the promoter as well as other gene sequences which may be necessary for the initiation of transcription. The presence of a promoter region is sufficient to cause the expression of an operably linked gene sequence.

“Rapid Acetylator Phenotype” is a characteristic of an individual in whom acetylation of amine containing compounds in the liver is rapid in comparison to other individuals. In determining this phenotype, various tests are conducted and described herein, including a caffeine test.

“Slow Acetylator Phenotype” is a characteristic of an individual in whom acetylation of amine containing compounds in the liver is slow in comparison to other individuals. In determining this phenotype, various tests are conducted and described herein including a caffeine test.

### SUMMARY OF INVENTION

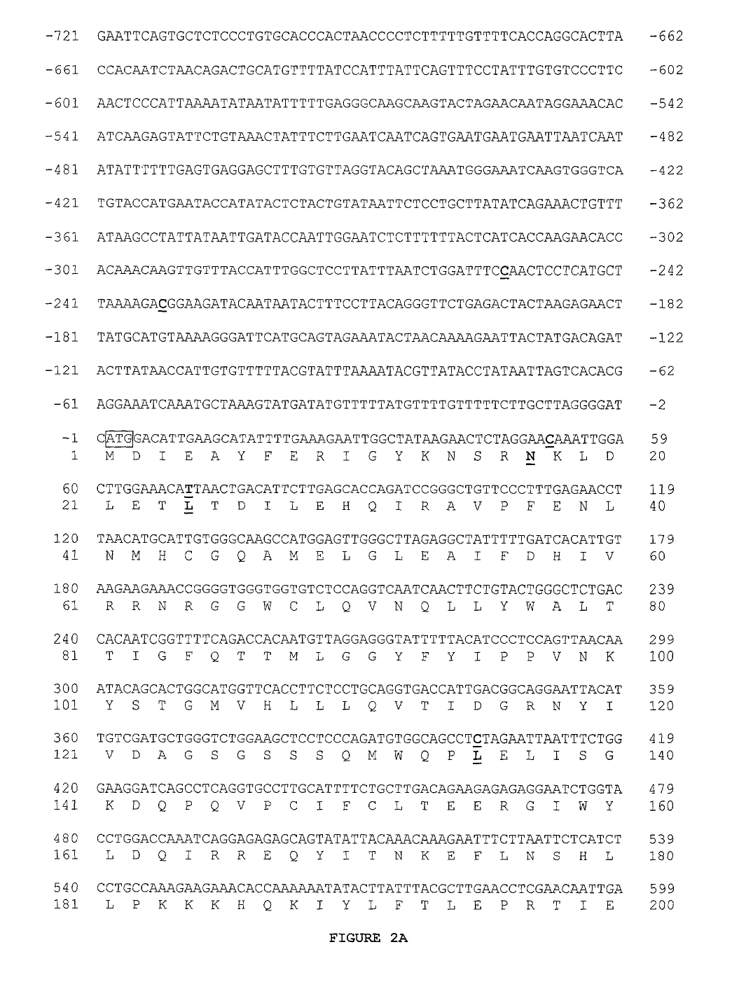

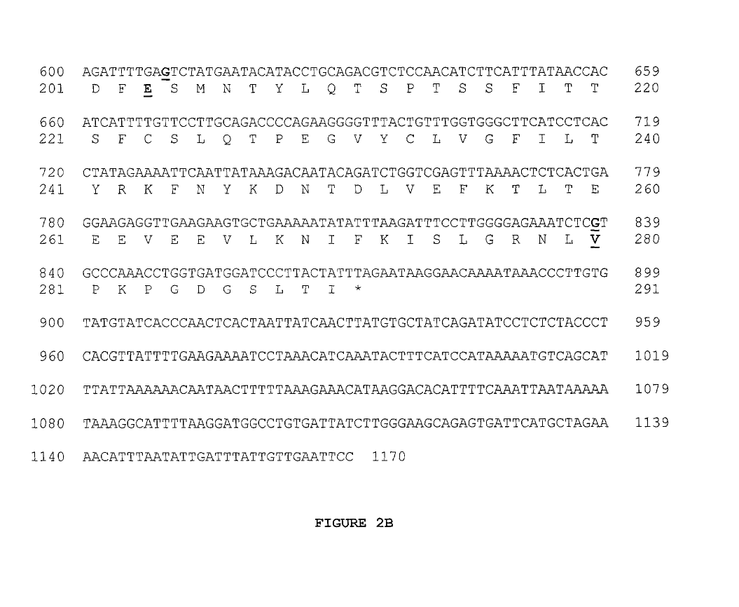

This invention includes nucleic acids sequences shown in FIGS. 2A-2B and Table 2, relating to polymorphic sites of the NAT-2 gene. The present invention further relates to polymorphisms as they exist within the general population and within various racial groups. Complements of these sequences are also included in this invention. The segments can be RNA or DNA, and can be single or double-stranded. The invention further relates to allele-specific oligonucleotides that hybridize to any of the sequences shown in FIGS. 2A-2B and Table 2. Vectors and host cells containing the nucleic acids herein described are also part of this invention.

Another embodiment includes a probe containing a polymorphism of Table 2. In yet another embodiment the invention provides an allele specific primer. The invention also provides a kit to identify individuals containing a NAT-2 polymorphism.

The nucleic acids of this invention can be used in therapeutic applications for a multitude of diseases either through the overexpression of a recombinant nucleic acid comprising all or a portion of a sequences disclosed in FIGS. 2A-2B and Table 2, or by the use of these oligonucleotides and genes to directly or indirectly modulate the expression of an endogenous gene or the activity of an endogenous gene product. Examples of therapeutic approaches include anti-sense inhibition of gene expression, gene therapy, antibodies that specifically bind to the gene products, and the like. Recombinant expression of the gene products in vitro is also a part of this invention.

In one embodiment, diagnostic methods which utilize all or part of the nucleic acids of this invention are described. Such nucleic acids can be used, for example, as part of diagnostic methods to identify NAT-2 polymorphisms of nucleic acids as a predisposition to various diseases including, but not limited to, bladder cancer, colon cancer, prostate cancer, Gilbert's disease, and leprosy.

A further embodiment includes a method of creating a prognosis protocol for a patient receiving a therapeutic composition metabolized by NAT-2 such as isoniazid, phenylzine, hydrazine, dapsone, procainamide, sulfamethazine and other sulfonamides. The method includes: a) identifying patients receiving one of these drugs, b) determining whether they are rapid acetylator or a slow acetylator; and c) converting the data obtained from step (b) into a prognosis protocol. The prognosis protocol may include prediction of drug efficacy, prediction of patient's prognosis, prediction of drug interaction, and prediction of adverse effects.

The invention also relates to the identification of differences among individuals in their metabolism of foreign compounds, including, but not limited to carcinogens or mutagens, including 2-aminofluorene, 4-aminobiphenyl, benzidine, beta-naphthylamine, and certain heterocyclic arylamines present in protein pyrolysates.

In a further embodiment, this invention describes the frequency of the polymorphisms of NAT-2 in different ethnic populations. Based on the this information, ethnic groups which are more susceptible to various diseases and disorders described above can be identified. Furthermore, this information can assist a physician in determining the best therapeutic composition for an individual from a specific ethnic group.

Another embodiment is a method to assist in development of therapeutic compositions through clinical trials. The method includes: a) administering a therapeutic composition to an individual and measuring its efficacy; b) determining by the individual's genotype and the SNPs provided herein, whether the individual is a rapid acetylator and slow acetylator; and c) determining from steps (a) and (b) which therapeutic composition will be the most effective for that particular genotype and which will have the least adverse effects.

Proteins, polypeptides, and peptides encoded by all or a part of the nucleic acids comprising NAT-2 nucleic acid sequences described in FIGS. 2A-2B and Table 2 are included in this invention. Such amino acid sequences are useful for diagnostic and therapeutic purposes. Further, antibodies can be raised against all or a part of these amino acid sequences for specific diagnostic and therapeutic methods requiring such antibodies. These antibodies can be polyclonal, monoclonal, or antibody fragments.

In a further embodiment, vectors and host cells containing vectors which comprise all or a portion of the nucleic acid sequences of this invention can be constructed for nucleic acid preparations, including anti-sense, and/or for expression of encoded proteins and polypeptides. Such host cells can be prokaryotic or eukaryotic cells. Further, the host cells can be part of tissue cultures or cell lines.

This invention also includes nonhuman transgenic animals, cells, cell lines or tissue cultures containing one or more of the nucleic acids of this invention useful for screening and for other purposes. Knockout nonhuman transgenic animals, cells, cell lines or tissue cultures can be produced wherein one or more endogenous genes or portions of such genes corresponding to the nucleic acids of this invention by function or structure are replaced by marker genes or are otherwise deleted in these cells, tissue culturs or animals. These modifications can result in cells or organisms which are heterozygous or homozygous for the deletion.

And yet another embodiment includes a computer readable medium comprising at least one nucleic acid sequence of Table 2.

### DETAILED DESCRIPTION OF THE INVENTION

This invention relates to seven novel NAT-2 gene polymorphisms. These polymorphisms occur in at least three ethnic groups. As described in Example 1, the claimed polymorphisms have been identified through polymerase chain reaction (PCR) and DNA sequencing techniques. The methodology described in Example 1 is not meant to be limiting. The detection of polymorphisms in specific DNA sequences, can be accomplished by a variety of methods including, but not limited to, restriction-fragment-length-polymorphism detection based on allele-specific restriction-endonuclease cleavage Kan and Dozy Lancet ii:910-912 (1978)), hybridization with allele-specific oligonucleotide probes (Wallace et al. Nucl Acids Res. 6:3543-3557 (1978)), including immobilized oligonucleotides (Saiki et al. Proc. Natl. Acad. Sd. USA 86:6230-6234 (1969)) or oligonucleotide arrays (Maskos and Southern Nucl Acids Res 21:2269-2270 (1993)), allele-specific PCR Newton et al. Nucl Acids Res 17:2503-2516 (1989)), mismatch-repair detection (MRD) (Faham and Cox Genome Res 5:474-482 (1995)), binding of MutS protein (Wagner et al. Nucl Acids Res 23:3944-3948 (1995), denaturing-gradient gel electrophoresis (DGGE) (Fisher and Lerman et al. Proc. Natl. Acad. Sci. USA. 80:1579-1583 (1983)), single-strand-conformation-polymorphism detection (Orita et al. Genomics 5:874-879 (1983)), RNAase cleavage at mismatched base-pairs (Myers et al. Science 230:1242 (1985)), chemical (Cotton et al. Proc. Natl. Acad. Sci. U.S.A, 8Z4397-4401(1988)) or enzymatic (Youil et al. Proc. Natl. Acad. Sci. U.S.A. 92:87-91(1995)) cleavage of heteroduplex DNA, methods based on allele specific primer extension (Syvanen et al. Genomics 8:684-692 (1990)), genetic bit analysis (GBA) Nikiforov et al. Nucl Acids 22:4167-4175 (1994)), the oligonucleotide-ligation assay (OLA) (Landegren et al. Science 241:1077 (1988)), the allele-specific ligation chain reaction (LCR) (Barrany Proc. Natl. Acad. Sci. U.S.A. 88:189-193 (1991)), gap-LCR (Abravaya et al. Nud Acids Res 23:675-682 (1995)), radioactive and/or fluorescent DNA sequencing using standard procedures well known in the art, and peptide nucleic acid (PNA) assays (Orum et al., Nuci. Acids Res, 21:5332-5356(1993).

The seven polymorphisms depicted in FIGS. 2A-2B and Table 2 include two in the 5′ non-coding region (C→G at base −255, and C→T at base −234) and five in the coding region (C→G at base 51, T→A at base 70, C→G at base 403, G→T at base 609, and G→A at base 838). These five mutations in the coding region change the amino acid transcribed at these positions (N→K at amino acid position 17, L→I at amino acid position 24, L→V at amino acid position 135, E→D at amino acid position 203, and V→M at amino acid position 280).

As described above, the present invention relates to NAT-2 nucleic acids comprising the corresponding cDNA sequences (FIGS. 2A-2B and Table 2), RNA, fragments of the genomic, cDNA, or RNA nucleic acids comprising 10, 15, 20, 25, 30, 35, 40, 50, 60, 70, 80, 90, 100, 200, 500 or more contiguous nucleotides, and the complements thereof. Closely related variants are also included as part of this invention, as well as recombinant nucleic acids comprising at least 50, 60, 70, 80, 90 or 95% of the nucleic acids described above which would be identical to the NAT-2 nucleic acids except for one or a few substitutions, deletions, or additions.

Further, the nucleic acids of this invention include the adjacent chromosomal regions of NAT-2 required for accurate expression of the respective gene. In a preferred embodiment, the present invention is directed to at least 15 contiguous nucleotides of the nucleic acid sequence of FIGS. 2A-2B and Table 2.

This invention further relates to methods using isolated and/or recombinant nucleic acids (DNA or RNA) that are characterized by their ability to hybridize to (a) a nucleic acid encoding a protein or polypeptide, such as a nucleic acid having any of the sequences of FIGS. 2A-2B and Table 2 or (b) a portion of the foregoing (e.g., a portion comprising the minimum nucleotides NAT-2 protein required to encode a functional NAT-2 protein; or by their ability to encode a polypeptide having the amino acid sequence of FIGS. 2A-2B and Table 2, or to encode functional equivalents thereof, e.g., a polypeptide which when incorporated into a cell, has all or part of the activity of a NAT-2 protein, or by both characteristics. A functional equivalent one of a NAT-2 proteins, therefore, would have a similar amino acid sequence (at least 65% sequence identity) and similar characteristics to, or perform in substantially the same way as one of the NAT-2 proteins. A nucleic acid which hybridizes to a nucleic acid encoding a NAT-2 protein or polypeptide, such as FIGS. 2A-2B and Table 2 can be double- or single-stranded. Hybridization to DNA such as DNA having the sequence of FIGS. 2A-2B and Table 2 includes hybridization to the strand shown or its complementary strand.

In one embodiment, the percent amino acid sequence similarity between a NAT-2 polypeptide such as FIGS. 2A-2B and Table 2, and functional equivalents thereof is at least about 50%. In a preferred embodiment, the percent amino acid sequence similarity between such a NAT-2 polypeptide and its functional equivalents is at least about 65%. More preferably, the percent amino acid sequence similarity between NAT-2 polypeptide and its functional equivalents is at least about 75%, and still more preferably, at least about 80%.

To determine percent nucleotide or amino acid sequence similarity, sequences can be compared to the publicly available unigene database (National Center for Biotechnology Information, National Library of Medicine, 38A, 8N905, 8600 Rockville Pike, Bethesda, Md. 20894; www.ncbi.nlm.nih.gov) using the blastn2 algorithm (Altschul, Nucl. Acids Res., 25:3389-3402 (1997)). The parameters for a typical search are: E=0.05, v=50, B=50 (where E is the expected probability score cutoff, V is the number of database entries returned in the reporting of the results, and B is the number of sequence alignments returned in the reporting of the results (Altschul et al, J. Mol. Biol., 215:403-410 (1990)).

Isolated and/or recombinant nucleic acids meeting these criteria comprise nucleic acids having sequences identical to sequences of naturally occurring NAT-2 genes and portions thereof, or variants of the naturally occurring genes. Such variants include mutants differing by the addition, deletion or substitution of one or more nucleotides, modified nucleic acids in which one or more nucleotides are modified (e.g., DNA or RNA analogs), and mutants comprising one or more modified nucleotides.

Such nucleic acids, including DNA or RNA, can be detected and isolated by hybridization under high stringency conditions or moderate stringency conditions, for example, which are chosen so as to not permit the hybridization of nucleic acids having non-complementary sequences. “Stringency conditions” for hybridizations is a term of art which refers to the conditions of temperature and buffer concentration which permit hybridization of a particular nucleic acid to another nucleic acid in which the first nucleic acid may be perfectly complementary to the second, or the first and second may share some degree of complementarity which is less than perfect. For example, certain high stringency conditions can be used which distinguish perfectly complementary nucleic acids from those of less complementarity. “High stringency conditions” and “moderate stringency conditions” for nucleic acid hybridizations are explained on pages 2.10.1-2.10.16 (see particularly 2.10.8-11) and pages 6.3.1-6 in Current Protocols in Molecular Biology (Ausubel, F. M. et al., eds., Vol. 1, containing supplements up through Supplement 29, 1995), the teachings of which are hereby incorporated by reference. The exact conditions which determine the stringency of hybridization depend not only on ionic strength, temperature and the concentration of destabilizing agents such as formamide, but also on factors such as the length of the nucleic acid sequence, base composition, percent mismatch between hybridizing sequences and the frequency of occurrence of subsets of that sequence within other non-identical sequences. Thus, high or moderate stringency conditions can be determined empirically.

High stringency hybridization procedures (1) employ low ionic strength and high temperature for washing, such as 0.015 M NaCl/0.0015 M sodium citrate, pH 7.0 (0.1×SSC) with 0.1% sodium dodecyl sulfate (SDS) at 50° C.; (2) employ during hybridization 50% (vol/vol) formamide with 5× Denhardt's solution (0.1% weight/volume highly purified bovine serum albumin/0.1% wt/vol Ficoll/0.1% wt/vol polyvinylpyrrolidone), 50 mM sodium phosphate buffer at pH 6.5 and 5×SSC at 42° C.; or (3) employ hybridization with 50% formamide, 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 0.1% SDS.

By varying hybridization conditions from a level of stringency at which no hybridization occurs to a level at which hybridization is first observed, conditions which will allow a given sequence to hybridize with the most similar sequences in the sample can be determined.

Exemplary conditions are described in Krause, M. H. and S. A. Aaronson (1991) Methods in Enzymology, 200:546-556. Also, see especially page 2, 10, 11 in Current Protocols in Molecular Biology (supra), which describes how to determine washing conditions for moderate or low stringency conditions. Washing is the step in which conditions are usually set so as to determine a minimum level of complementarity of the hybrids. Generally, from the lowest temperature at which only homologous hybridization occurs, a 1% mismatch between hybridizing nucleic acids results in a 1° C. decrease in the melting temperature Tₘ, for any chosen SSC concentration. Generally, doubling the concentration of SSC results in an increase in Tₘ of ˜117° C. Using these guidelines, the washing temperature can be determined empirically for moderate or low stringency, depending on the level of mismatch sought.

Isolated and/or recombinant nucleic acids that are characterized by their ability to hybridize to (a) a nucleic acid encoding a NAT-2 polypeptide, such as the nucleic acids depicted FIGS. 2A-2B and Table 2 (b) the complement of FIGS. 2A-2B and Table 2, (c) or a portion of (a) or (b) (e.g. under high or moderate stringency conditions), may further encode a protein or polypeptide having at least one function characteristic of a NAT-2 polypeptide, such as N-acetylation, or binding of antibodies that also bind to non-recombinant NAT-2 protein or polypeptide. The catalytic or binding function of a protein or polypeptide encoded by the hybridizing nucleic acid may be detected by standard enzymatic assays for activity or binding (e.g., assays which measure the binding of a transit peptide or a precursor, or other components of the translocation machinery). Enzymatic assays, complementation tests, or other suitable methods can also be used in procedures for the identification and/or isolation of nucleic acids which encode a polypeptide such as a polypeptide of the amino acid sequence FIGS. 2A-2B and Table 2, or a functional equivalent of this polypeptide. The antigenic properties of proteins or polypeptides encoded by hybridizing nucleic acids can be determined by immunological methods employing antibodies that bind to a NAT-2 polypeptide such as immunoblot, immunoprecipitation and radioimmunoassay. PCR methodology, including RAGE (Rapid Amplification of Genomic DNA Ends), can also be used to screen for and detect the presence of nucleic acids which encode NAT-2-like proteins and polypeptides, and to assist in cloning such nucleic acids from genomic DNA. PCR methods for these purposes can be found in Innis, M. A., et al. (1990) PCR Protocols: A Guide to Methods and Applications, Academic Press, Inc., San Diego, Calif., incorporated herein by reference.

It is understood that, as a result of the degeneracy of the genetic code, many nucleic acid sequences are possible which encode a NAT-2-like protein or polypeptide. Some of these will have little homology to the nucleotide sequences of any known or naturally-occurring NAT-2-like gene but can be used to produce the proteins and polypeptides of this invention by selection of combinations of nucleotide triplets based on codon choices. Such variants, while not hybridizable to a naturally-occurring NAT-2 gene, are contemplated within this invention.

The nucleic acids described herein are used in the methods of the present invention for production of proteins or polypeptides, through incorporation into cells, tissues, or organisms. In one embodiment, DNA containing all or part of the coding sequence for a NAT-2 polypeptide, or DNA which hybridizes to DNA having the sequence in FIGS. 2A-2B and Table 2, is incorporated into a vector for expression of the encoded polypeptide in suitable host cells. The encoded polypeptide consisting of NAT-2, or its functional equivalent is capable of normal activity, such as N-acetylation. The term “vector” as used herein refers to a nucleic acid molecule capable of transporting another nucleic acid to which it has been linked. A vector, for example, can be a plasmid.

Nucleic acids referred to herein as “isolated” are nucleic acids separated away from the nucleic acids of the genomic DNA or cellular RNA of their source of origin (e.g., as it exists in cells or in a mixture of nucleic acids such as a library), and may have undergone further processing. “Isolated”, as used herein, refers to nucleic or amino acid sequences that are at least 60% free, prefereably 75% free, and most preferably 90% free from other components with which they are naturally associated. “Isolated” nucleic acids (polynucleotides) include nucleic acids obtained by methods described herein, similar methods or other suitable methods, including essentially pure nucleic acids, nucleic acids produced by chemical synthesis, by combinations of biological and chemical methods, and recombinant nucleic acids which are isolated. Nucleic acids referred to herein as “recombinant” are nucleic acids which have been produced by recombinant DNA methodology, including those nucleic acids that are generated by procedures which rely upon a method of artificial recombination, such as the polymerase chain reaction (PCR) and/or cloning into a vector using restriction enzymes. “Recombinant” nucleic acids are also those that result from recombination events that occur through the natural mechanisms of cells, but are selected for after the introduction to the cells of nucleic acids designed to allow or make probable a desired recombination event. Portions of the isolated nucleic acids which code for polypeptides having a certain function can be identified and isolated by, for example, the method of Jasin, M., et al., U.S. Pat. No. 4,952,501.

The invention also relates to proteins or polypeptides encoded by the novel nucleic acids described herein. The proteins and polypeptides of this invention can be isolated and/or recombinant. Proteins or polypeptides referred to herein as “isolated” are proteins or polypeptides purified to a state beyond that in which they exist in cells. In a preferred embodiment, they are at least 10% pure; i.e., most preferably they are substantially purified to 80 or 90% purity. “Isolated” proteins or polypeptides include proteins or polypeptides obtained by methods described infra, similar methods or other suitable methods, and include essentially pure proteins or polypeptides, proteins or polypeptides produced by chemical synthesis or by combinations of biological and chemical methods, and recombinant proteins or polypeptides which are isolated. Proteins or polypeptides referred to herein as “recombinant” are proteins or polypeptides produced by the expression of recombinant nucleic acids.

In a preferred embodiment, the protein or portion thereof has at least one function characteristic of a NAT-2 protein or polypeptide, for example, N-acetylation, and/or antigenic function (e.g., binding of antibodies that also bind to naturally occurring NAT-2 polypeptide). As such, these proteins are referred to as analogs, and include, for example, naturally occurring NAT-2, variants (e.g. mutants) of those proteins and/or portions thereof. Such variants include mutants differing by the addition, deletion or substitution of one or more amino acid residues, or modified polypeptides in which one or more residues are modified, and mutants comprising one or more modified residues. The variant can have “conservative” changes, wherein a substituted amino acid has similar structural or chemical properties, e.g., replacement of leucine with isoleucine. More infrequently, a variant can have “nonconservative” changes, e g., replacement of a glycine with a tryptophan. Guidance in determining which amino acid residues can be substituted, inserted, or deleted without abolishing biological or immunological activity can be found using computer programs well known in the art, for example, DNASTAR. software (DNASTAR, Inc., Madison, Wis. 53715 U.S.A.).

A “portion” as used herein with regard to a protein or polypeptide, refers to fragments of that protein or polypeptide. The fragments can range in size from 5 amino acid residues to all but one residue of the entire protein sequence. Thus, a portion or fragment can be at least 5, 5-50, 50-100, 100-200, 200-400, 400-800, or more consecutive amino acid residues of a NAT-2 protein or polypeptide, for example, FIG. 2 and Table 2, or a variant thereof.

The invention also relates to isolated, synthesized and/or recombinant portions or fragments of a NAT-2 protein or polypeptide as described above. Polypeptide fragments of the enzyme can be made which have full or partial function on their own, or which when mixed together (though fully, partially, or nonfunctional alone), spontaneously assemble with one or more other polypeptides to reconstitute a functional protein having at least one functional characteristic of a NAT-2 protein of this invention.

The invention also concerns the use of the nucleotide sequence of the nucleic acids of this invention to identify DNA probes for NAT-2 genes, PCR primers to amplify NAT-2 genes, and regulatory elements of the NAT-2 genes.

Preparation of Nucleic Acids, Vectors Transformations and Host Cells

DNA fragments can be prepared, for example, by digesting plasmid DNA, or by use of PCR. Oligonucleotides for use as primers or probes are chemically synthesized by methods known in the field of the chemical synthesis of polynucleotides, including by of non-limiting example the phosphoramidite method described by Beaucage and Carruthers, Tetrahedron Lett 22.1859-1 862 (1981) and the triester method provided by Matteucci, et al J Am. Chem. Soc. 103:3185 (1981) both incorporated herein by reference. These syntheses may employ an automated synthesizer, as described in Needham-VanDevanter, D. R., et al., Nucleic Acids Res. 12:61596168(1984). Purification of oligonucleotides may be carried out by either native acrylamide gel electrophoresis or by anion-exchange HPLC as described in Pearson, J. D. and Regnier, F E.,, J. Chrom,, 255:137-149(1983). A double stranded fragment may then be obtained, if desired, by annealing appropriate complementary single strands together under suitable conditions or by synthesizing the complementary strand using a DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

The sequence of the synthetic oligonucleotide or of any nucleic acid fragment can be can be obtained using either the dideoxy chain termination method or the Maxam-Gilbert method (see Sambrook et al. Molecular Cloning—a Laboratory Manual (2nd Ed.), Vols. 1-3, Cold Spring Harbor Laboratory, Cold Spring Harbor, N.Y., (1989), which is incorporated herein by reference. This manual is hereinafter referred to as “Sambrook et al.”; Zyskind et al., (1988)). Recombinant DNA Laboratory Manual, (Acad. Press, New York). Oligonucleotides useful in diagnostic assays are typically at least 8 consecutive nucleotides in length, and may range upwards of 18 nucleotides in length to greater than 100 or more consecutive nucleotides.

Nucleic acid constructs prepared for introduction into a prokaryotic or eukaryotic host will comprise a replication system recognized by the host, including the intended nucleic acid fragment encoding the selected protein or polypeptide, and will preferably also include transcription and translational initiation regulatory sequences operably linked to the protein encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals are also included, where appropriate, whether from a native NAT-2 protein or from other receptors or from secreted proteins of the same or related species, which allow the protein to cross and/or lodge in cell membranes, and thus attain its functional topology, or be secreted from the cell. Such vectors may be prepared by means of standard recombinant techniques well known in the art and discussed, for example, in Sambrook et al, Molecular Cloning. A Laboratory Manual, 2nd Ed. (Cold Spring Harbor Laboratory, Cold Spring Harbor, N.Y. (1989) or Ausubel et al, Current Protocols in Molecular Biology, J. Wiley and Sons, NY (1992).

An appropriate promoter and other necessary vector sequences will be selected so as to be functional in the host, and will include, when appropriate, those naturally associated with NAT-2 genes. Examples of workable combinations of cell lines and expression vectors are described in Sambrook et al, Molecular Cloning. A Laboratory Manual, 2nd Ed. (Cold Spring Harbor Laboratory, Cold Spring Harbor, N.Y. (1989) or Ausubel et al, Current Protocols in Molecular Biology, J. Wiley and Sons, NY (1992). Many useful vectors are known in the art and can be obtained from such vendors as Stratagene (supra), New England BioLabs, Beverly, Me., U.S.A, Promega Biotech, and other biotechnology product suppliers. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. Vectors and promoters suitable for use in yeast expression are further described in EP 73,675A. Appropriate non-native mammalian promoters might include the early and late promoters from SV40 (Fiers et al, Nature, 273:113 (1978)) or promoters derived from murine Moloney leukemia virus, mouse tumor virus, avian sarcoma viruses, adenovirus II, bovine papilloma virus or polyoma. In addition, the construct may be joined to an amplifiable gene (e.g., DHFR) so that multiple copies of the gene may be made. For appropriate enhancer and other expression control sequences, see also Enhancers and Eukaryotic Gene Expression, Cold Spring Harbor Press, Cold Spring Harbor, N.Y. (1983). While such expression vectors may replicate autonomously, they may also replicate by being inserted into the genome of the host cell, by methods well known in the art.

Expression and cloning vectors will likely contain a selectable marker, a gene encoding a protein necessary for survival or growth of a host cell transformed with the vector. The presence of this gene ensures growth of only those host cells which express the inserts. Typical selection genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

The vectors containing the nucleic acids of interest can be transcribed in vitro, and the resulting RNA introduced into the host cell by well-known methods, e.g., by injection (see, Kubo et al, FEBS Letts. 241:119 (1988)), or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. See generally, Sambrook et al., 1989 and Ausubel et al., 1992. The introduction of the nucleic acids into the host cell by any method known in the art, including those described above, will be referred to herein as “transformation.” The cells into which have been introduced nucleic acids described above are meant to also include the progeny of such cells.

Large quantities of the nucleic acids and proteins of the present invention may be prepared by expressing the NAT-2 nucleic acids or portions thereof in vectors or other expression vehicles in compatible prokaryotic or eukaryotic host cells. The most commonly used prokaryotic hosts are strains of Escherichia coli, although other prokaryotes, such as Bacillus subtilis or Pseudomonas may also be used.

Mammalian or other eukaryotic host cells, such as those of yeast, filamentous fungi, plant, insect, or amphibian or avian species, may also be useful for production of the proteins of the present invention. Propagation of mammalian cells in culture is per se well known. See, Jakoby and Pastan (eds.), Cell Culture. Methods in Enzymology, volume 58, Academic Press, Inc., Harcourt Brace Jovanovich, N.Y., (1979)). Examples of commonly used mammalian host cell lines are VERO and HeLa cells, Chinese hamster ovary (CHO) cells, and WI38, BHK, and COS cell lines, although it will be appreciated by the skilled practitioner that other cell lines may be appropriate, e.g., to provide higher expression desirable glycosylation patterns, or other features.

Clones are selected by using markers depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

Prokaryotic or eukaryotic cells transformed with the nucleic acids of the present invention will be useful not only for the production of the nucleic acids and proteins of the present invention, but also, for example, in studying the characteristics of NAT-2 proteins.

Allele Specific Primers and Oligonucleotides

The invention further provides nucleotide primers which can detect polymorphisms of the invention. According to another aspect of the present invention there is provided an allele specific primer capable of detecting a NAT-2 polymorphism at one or more of positions −255,−234, 51, 70, 403, 609, and 838 in the NAT-2 gene as defined by the positions in Table 2 and FIGS. 2A-2B.

An allele specific primer is used, generally together with a constant primer, in an amplification reaction such as a PCR reaction, which provides the discrimination between alleles through selective amplification of one allele at a particular sequence position e g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′, terminus correspond with the allele to be detected and wherein up to 10, such as up to 8, 6, 4, 2 or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labeled to facilitate detection.

According to another aspect of the present invention there is provided an allele-specific oligonucleotide probe capable of detecting a NAT-2 polymorphism at one or more of positions −255,− 234, 51, 70, 403, 609, and 838 in the NAT-2 gene as defined by the positions in Table 2 and FIGS. 2A-2B.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s), nucleotides, and polymerase(s) such as thermostable polymerases, for example taq polymerase.

Protein Expression and Purification

The invention also relates to polypeptide sequences of Table 2 and FIGS. 2A-2B. The polypeptide can contain 5 amino acid bases, more preferably 10 bases. Once DNA encoding a sequence comprising a SNP is isolated and cloned, one can express the encoded polymorphic proteins in a variety of recombinantly engineered cells. It is expected that those of skill in the art are knowledgeable in the numerous expression systems available for expression of DNA encoding a sequence of interest. No attempt to describe in detail the various methods known for the expression of proteins in prokaryotes or eukaryotes is made here.

In brief summary, the expression of natural or synthetic nucleic acids encoding a sequence of interest will typically be achieved by operably linking the DNA or cDNA to a promoter (which is either constitutive or inducible), followed by incorporation into an expression vector. The vectors can be suitable for replication and integration in either prokaryotes or eukaryotes. Typical expression vectors contain, initiation sequences, transcription and translation terminators, and promoters useful for regulation of the expression of a polynucleotide sequence of interest. To obtain high level expression of a cloned gene, it is desirable to construct expression plasmids which contain, at the minimum, a strong promoter to direct transcription, a ribosome binding site for translational initiation, and a transcription/translation terminator. The expression vectors may also comprise generic expression cassettes containing at least one independent terminator sequence, sequences permitting replication of the plasmid in both eukaryotes and prokaryotes. i.e., shuttle vectors, and selection markers for both prokaryotic and eukaryotic syszems. See Sambrook et al.

A variety of prokaryotic expression systems may be used to express the polymorphic proteins of the invention. Examples include E. coli, Bacillus, Streptomyces, and the like.

It is preferred to construct expression plasmids which contain, at the minimum, a strong promoter to direct transcription, a ribosome binding site for translational initiation, and a transcription/translation terminator. Examples of regulatory regions suitable for this purpose in E. coli are the promoter and operator region of the E. coli tryptophan biosynthetic pathway as described by Yanofsky, C., J. Bacterial. 158:1018-1024(1984) and the leftward promoter of phage lambda (P) as described by A. I. and Hagen, D. Ann. Rev. Genet. 14:399-445 (1980). The inclusion of selection markers in DNA vectors transformed in E. coli is also useful. Examples of such markers include genes specifying resistance to ampicillin, tetracycline, or chloramphenicol. See Sambrook et al. for details concerning selection markers for use in E. coli.

To enhance proper folding of the expressed recombinant protein, during purification from E. coli, the expressed protein may first be denatured and then renatured. This can be accomplished by solubilizing the bacterially produced proteins in a chaotropic agent such as guanidine HCl and reducing all the cysteine residues with a reducing agent such as beta-mercaptoethanol. The protein is then renatured, either by slow dialysis or by gel filtration. See U.S. Pat. No. 4,511,503. Detection of the expressed antigen is achieved by methods known in the art as radioimmunoassay, or Western blotting techniques or immunoprecipitation. Purification from E. coli can be achieved following procedures such as those described in U.S. Pat. No.4,511,503.

Any of a variety of eukaryotic expression systems such as yeast, insect cell lines, bird, fish, and mammalian cells, may also be used to express a polymorphic protein of the invention. As explained briefly below, a nucleotide sequence harboring a SNP may be expressed in these eukaryotic systems. Synthesis of heterologous proteins in yeast is well known. Methods in Yeast Genetics, Sherman, F., et al., Cold Spring Harbor Laboratory, (1982) is a well recognized work describing the various methods available to produce the protein in yeast. Suitable vectors usually have expression control sequences, such as promoters, including 3-phosphogtycerate kinase or other glycolytic enzymes, and an origin of replication, termination sequences and the like as desired. For instance, suitable vectors are described in the literature (Botstein, et al.,Gene 8:17-24 (1979); Broach, et al., Gene 8:121-133 (1979)).

Two procedures are used in transforming yeast cells. In one case, yeast cells are first converted into protoplasts using zymolyase, lyticase or glusulase, followed by addition of DNA and polyethylene glycol (PEG). The PEG-treated proloplasts are then regenerated in a 3% agar medium under selective conditions. Details of this procedure are given in the papers by J. D. Beggs, Nature (London) 275:104-109 (1978); and Hinnen, A., et al., Proc. Nati. Acad. Sci. USA, 75:1929-1933 (1978). The second procedure does not involve removal of the cell wall. Instead the cells are treated with lithium chloride or acetate and PEG and put on selective plates (Ito, H., et al., J. Bact, 153163-168 (1983)). cells and applying standard protein isolation techniques to the lysates.

The purification process can be monitored by using Western blot techniques or radio immunoassay or other standard techniques. The sequences encoding the proteins of the invention can also be ligated to various immunoassay expression vectors for use in transforming cell cultures of, for instance, mammalian, insect, bird or fish origin. Illustrative of cell cultures useful for the production of the polypeptides are mammalian cells. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines, and various human cells such as COS cell lines, HeLa cells, myeloma cell lines, Jurkat cells, etc. Expression vectors for these cells can include expression control sequences, such as an origin of replication, a promoter (e.g., the CMV promoter, a HSV tk promoter or pgk (phosphoglycerate kinase) promoter), an enhancer (Queen et al. Immunol. Rev.89:49 (1986)) and necessary processing information sites, such as ribosome binding sites, RNA splice sites, polyadenylation sites (e.g., an SV40large T Ag poly A addition site), and transcriptional terminator sequences.

Other animal cells are available, for instance, from the American Type Culture Collection Catalogue of Cell Lines and Hybridomas (7th edition, (1992)). Appropriate vectors for expressing the proteins of the invention in insect cells are usually derived from baculovirus. Insect cell lines include mosquito larvae, silkworm, armyworm, moth and Drosophila cell lines such as a Schneider cell line (See Schneider J. Embryol. Exp. Morphol., 27:353-365 (1987). As indicated above, the vector, e.g., a plasmid, which is used to transform the host cell, preferably contains DNA sequences to initiate transcription and sequences to control the translation of the protein. These sequences are referred to as expression control sequences. As with yeast, when higher animal host cells are employed, polyadenylation or transcription terminator sequences from known mammalian genes need to be incorporated into the vector. An example of a terminator sequence is the polyadenylation sequence from the bovine growth hormone gene. Sequences for accurate splicing of the transcript may also be included. An example of a splicing sequence is the VPI intron from SV40(Sprague, J. et al., J. Virol. 45: 773-781 (1983)). Additionally, gene sequences to control replication in the host cell may be Saveria Campo, M., 1985, “Bovine Papilloma virus DNA a Eukaryotic Cloning Vector” in DNA Cloning Vol.11 a Practical AnDroach Ed. D. M. Glover, IRL Press, Arlington, Va. pp. 213-238. The host cells are competent or rendered competent for transformation by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation and micro-injection of the DNA directly into the cells.

The transformed cells are cultured by means well known in the art (Biochemical Methods in Cell Culture and Virology, Kuchler, R. J., Dowden, Hutchinson and Ross, Inc., (1977)). The expressed polypeptides are isolated from cells grown as suspensions or as monolayers. The latter are recovered by well known mechanical, chemical or enzymatic means.

General methods of expressing recombinant proteins are also known and are exemplified in R. Kaufman, Methods in Enzymology 185, 537-566 (1990). As defined herein “operably linked” refers to linkage of a promoter upstream from a DNA sequence such that the promoter mediates transcription of the DNA sequence. Specifically, “operably linked” means that the isolated polynucleotide of the invention and an expression control sequence are situated within a vector or cell in such a way that the gene encoding the protein is expressed by a host cell which has been transformed (transfected) with the ligated polynucleotide/expression sequence. The term “vector”, refers to viral expression systems, autonomous self-replicating circular DNA (plasmids), and includes both expression and nonexpression plasmids.

A number of types of cells may act as suitable host cells for expression of the protein. Mammalian host cells include, for example. monkey COS cells, Chinese Hamster Ovary (CHO) cells, Human kidney 293 cells, human epdiermal A431 cells, human Co10205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells. Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida or any yeast strain capable of expressing heterologous proteins. Potentially suitable bacteral strains include Escherichia coli, Bacillus sublilis, Salmonella typhimuri urn, or any bacterial strain capable of expressing heterologous proteins. If the protein is made in yeast or bacteria, it may be necessary to modify the protein produced therein, for example by phosphorylation or glycosyjation of the appropriate sites, in order to obtain the functional protein.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBacOc kit), and such methods are well known in the art, as described in Summers and Smith, Texas Agricultural Experiment Station Bulletin No. 1555 (1987). incorporated herein by reference. As used herein, an insect cell capable of expressing a polynucleotide of the present invention is “transformed.” The protein of the invention may be prepared by culturing transformed host cells under culture conditions suitable to express the recombinant protein.

The polymorphic protein of the invention may also be expressed as a product of transgenic animals, e.g., as a component of the milk of transgenic cows, goats, pigs, or sheep which are characterized by somatic or germ cells containing a nucleotide seqaence encoding the protein. The protein may also be produced by known conventional chemical synthesis. Methods for constructing the proteins of the present invention by synthetic means are known to those skilled in the art.

The polymorphic proteins produced by recombinant DNA technology may be purified by techniques commonly employed to isolate or purify recombinant proteins. Recombinantly produced proteins can be directly expressed or expressed as a fusion protein. The protein is then purified by a combination of cell lysis (e.g. sonication) and affinity chromatography. For fusion products, subsequent digestion of the fusion protein with an appropriate proteolytic enzyme releases the desired polypeptide. The polypeptides of this invention may be purified to substantial purity by standard techniques well known in the art, including selective precipitation with such substances as ammonium sulfate, column chromatography, immunopurification methods, and others. See, for instance, R. Scopes, Protein Purification: Principles and Practice, Springer-Verlag: New York (1982), incorporated herein by reference. For example, in an embodiment, antibodies may be raised to the proteins of the invention as described herein. Cell membranes are isolated from a cell line expressing the recombinant protein, the protein is extracted from the membranes and immunoprecipitated. The proteins may then be further purified by standard protein chemistry techniques as described above.

The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-Toyopearl or Cibacrom blue 3GA Sepharose B; one or more steps involving hydrophobic interaction chromatography using such resins as phenyl ether, butyl ether, or propyl ether; or immuno affinity chromatography. Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLab (Beverly, Me.), Pharmacia (Piscataway, N.J.) and InVitrogen, respectively. The protein can also be tagged with an epitope and subsequently purified by using a specific antibody directed to such epitope. One such epitope (“Flag”) is commercially available from Kodak New Haven, Conn.). Finally, one or more reverse-phase high performance liquid chromatography (RI)-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

Antibodies

The term “antibody” as used herein refers to immunoglobulin molecules and immunologically active portions of immunoglobulin molecules, i.e., molecules that contain an antigen binding site that specifically binds (immunoreacts with) an antigen, such as polymorphic. Such antibodies include, but are not limited to, polyclonal, monoclonal, chimeric, single chain, Fab and F(ab′)2 fragments, and an Fab expression library. In a specific embodiment, antibodies to human polymorphic proteins are disclosed.

The phrase “specifically binds to”, “immunospecifically binds to” or is “specifically immunoreactive with”, an antibody when referring to a protein or peptide, refers to a binding reaction which is determinative of the presence of the protein in the presence of a heterogeneous population of proteins and other biological materials. Thus, for example, under designated immunoassay conditions, the specified antibodies bind to a particular protein and do not bind in a significant amount to other proteins present in the sample. Specific binding to an antibody under such conditions may require an antibody that is selected for its specificity for a particular protein. Of particular interest in the present invention is an antibody that binds immunospecifically to a polymorphic protein but not to its cognate wild type allelic protein, or vice versa. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, a Laboratory Manual, Cold Spring Harbor Publications, New York, for a description of immunoassay formats and conditions that can be used to determine specific immunoreactivity.

Polyclonal and/or monoclonal antibodies that immunospecifically bind to polymorphic gene products but not to the corresponding prototypical or “wild-type” gene products are also provided. Antibodies can be made by injecting mice or other animals with the variant gene product or synthetic peptide. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product.

An isolated polymorphic protein, or a portion or fragment thereof; can be used as an immunogen to generate the antibody that bind the polymorphic protein using standard techniques for polyclonal and monoclonal antibody preparation. The full-length polymorphic protein can be used or, alternatively, the invention provides antigenic peptide fragments of polymorphic for use as immunogens. The antigenic peptide of a polymorphic protein of the invention comprises at least 5 amino acid residues of the amino acid sequence encompassing the polymorphic amino acid and encompasses an epitope of the polymorphic protein such that an antibody raised against the peptide forms a specific immune complex with the polymorphic protein. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of polymorphic that are located on the surface of the protein, e.g., hydrophilic regions.

For the production of polyclonal antibodies, various suitable host animals (e.g., rabbit, goat, mouse or other mammal) may be immunized by injection with the polymorphic protein. An appropriate immunogenic preparation can contain, for example, recombinantly expressed polymorphic protein or a chemically synthesized polymorphic polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Cozynebacterium parvum, or similar immunostimulatory agents. If desired, the antibody molecules directed against polymorphic proteins can be isolated from the mammal (e.g., from the blood) and flirther purified by well known techniques, such as protein A chromatography, to obtain the IgG fraction.

The term “monoclonal antibody” or “monoclonal antibody composition”, as used herein, refers to a population of antibody molecules that originates from the clone of a singly hybridoma cell, and that contains only one type of antigen binding site capable of immunoreacting with a particular epitope of a polymorphic protein. A monoclonal antibody composition thus typically displays a single binding affinity for a particular polymorphic protein with which it immunoreacts. For preparation of monoclonal antibodies directed towards a particular polymorphic protein, or derivatives, fragments, analogs or homologs thereof; any technique that provides for the production of antibody molecules by continuous cell line culture may be utilized. Such techiuques include, but are not limited to, the hybridoma technique (see Kohler & Milstein, 1975 Nature 256. 495-497); the trioma technique; the human B-cell hybridoma technique (see Kozbor, et al., 1983 immunol Today 4: 72) and the EBV hybridoma technique to produce human monoclonal antibodies (see Cole, et aL, 1985 In: MONOCLONAL ANTIBODIES AND CANCER THERAPY, Alan R. Liss, Inc., pp.77-96). Human monoclonal antibodies may be utilized in the practice of the present invention and may be produced by using human bybridomas (see Cote et al., 1983. Proc NatlAcadSci USA go: 2026-2030) or by transforming human B-cells with Epstein Barr Virus in vitro (see Cole, ef aL, I 985 In: MONOCLONAL ANTIBODIES AND CANCER THERAPY, Alan R. Liss, Inc., pp.77-96).

According to the invention, techniques can be adapted for the production of single-chain antibodies specific to a polymorphic protein (see e.g., U.S. Pat. No. 4,946,778). in addition, methodologies can be adapted for the construction of Fab expression libraries (see e.g., Huse, et al., 1989 Science 246:1275-1281) to allow rapid and effective identification of monoclonal Fab fragments with the desired specificity for a polymorphic protein or derivatives, fragments, analogs or homologs thereof. Non-human antibodies can be “humanized” by techniques well known in the art. See e.g., U.S. Pat. No. 5,225,539. Antibody fragments that contain the idiotypes to a polymorphic protein may be produced by techniques known in the art including, but not limited to: (i) an F(ab′)2 fragment produced by pepsin digestion of an antibody molecule; (ii) an Fab fragment generated by reducing the disuWide bridges of an F(ab)₂ fragment; (iii) an Fab fragment generated by the treatment of the antibody molecule with papain and a reducing agent and (iv) Fv fragments.

Additionally, recombinant anti-polymorphic protein antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/U586102269; European Patent Application No.184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) PNAS 84:3439-3443; Liu et al. (1987)] immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988)] Natl Cancer Inst 80:1553-1559); Morrison(I 985) Science 229:1202-1207; Oi etal. (1986) BioTechniques 4:214; U.S. Pat. No.5,225,539, Jones etal. (1986) Nature 321:552-525; Verhoeyan et al. (1988) Science 239:1534; and Beidler et al. (1988) J Immunol 141:4053-4060. In one embodiment, methodologies for the screening of antibodies that possess the desired specificity include, but are not limited to, enzyme-linked immunosorbent assay (ELISA) and other immunologically-mediated techniques known within the art.

Antisense Nucleic Acid Molecules

Another aspect of the invention pertains to isolated antisense nucleic acid molecules that are hybridizable to or complementary to the nucleic acid molecule comprising the SNP-containing nucleotide sequences of the invention, or fragments, analogs or derivatives thereof. An “antisense” nucleic acid comprises a nucleotide sequence that is complementary to a “sense” nucleic acid encoding a protein, e.g., complementary to the coding strand of a doable-stranded cDNA molecule or complementary to an mRNA sequence. In specific aspects, antisense nucleic acid molecules are provided that comprise a sequence complementary to at least about 10, about 25, about 50, or about 60 nucleotides or an entire SNP coding strand, or to only a portion thereof.

In one embodiment, an antisense nucleic acid molecule is antisense to a “coding region” of the coding strand of a polymorphic nucleotide sequence of the invention. The term “coding region” refers to the region of the nucleotide sequence comprising codons which are translated into amino acid. In another embodiment. the antisense nucleic acid molecule is antisense to a “noncoding region” of the coding strand of a nucleotide sequence of the invention. The term “noncoding region” refers to 5′ and 3′ sequences which flank the coding region that are not translated into amino acids (i.e., also referred to as 5′ and 3′ untranslated regions).

Given the coding strand sequences disclosed herein, antisense nucleic acids of the invention can be designed according to the rules of Watson and Crick or Hoogsteen base pairing. For example, the antisense nucleic acid molecule can generally be complementary to the entire coding region of an mRNA, but more preferably as embodied herein, it is an oligonucleotide that is antisense to only a portion of the coding or noncoding region of the mRNA. An antisense oligonucleotide can range in length between about 5 and about 60 nucleotides, preferably between about 10 and about 45 nucleotides, more preferably between about 15 and 40 nucleotides, and still more preferably between about 15 and 30 in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouraci I, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, I-methylguanine, I-methylinosine, 2,2-dimethylguanine, 2-methyladenine, 2-methylguanine, 3-metbylcytosine, 5-methylcytosine, N6-adenine, 7-methylguanine, 5-methylaminomethyluracil, 5-methoxyaminomethy-2-thiouracil, beta-D-mannosylqueosine, 5′-methoxycarboxymethyluracil, 5-methoxyuracil, 2-methyltbio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subdoned in an antisense orientation (i.e., RNA transcribed from the inserted nucleic acid will be of an anti sense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polymorphic protein to thereby inhibit expression of the protein, e.g., by inhibiting transcription and/or translation. The hybridization can be by conventional nucleotide complementary to form a stable duplex, or, for example, in the case of an anti sense nucleic acid molecule that binds to DNA duplexes, through specific interactions in the major groove of the double helix. An example of a route of administration of anti sense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of anti sense molecules, vector constructs in which the antisense nucleic acid molecule is placed under the control of a strong p0111 or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual-units, the strands run parallel to each other (Gaultier et al. (1987) Nucleic Acids Res 15: 6625-6641). T antisense nucleic acid molecule can also comprise a 2′-o-methylribonucleotide (lnoue et al. (1987) NucleicAcids Res 15:6131-6148) or a chimeric RNA-DNA analogue (Inoue et al. (1987) FEBS Lett215: 327-330).

Determining Phenotype

The nucleic acid sequences provided in Table 2 and FIGS. 2A-2B can be used to screen additional individuals and determine their respective phenotype as either slow or rapid acetylator. As described in Example 1, DNA can isolated from individuals and using DNA sequencing techniques known in the art, one can sequence the individual's NAT-2 gene. In particular, the SNPs provided by the inventors can be used to compare and confirm any polymorphisms from the individual. Once the polymorphisms are determined, the phenotype can then be correlated to that particular genotype.

Cascorbi et al. teaches a method of determining an individual's phenotype using a caffeine test (Am. J. Hum. Genet. 57: 581-592 (1995)). Briefly, 5 hours after ingesting a cup of coffee or a half a tablet of caffeine (Coffeinum 0.2 g compretten N, Cascan), urine is collected from an individual. Using various purification methods known in the art, the ratio of caffeine's secondary metabolites, 5-acetylamino-6-formylamino-3-methyl-uracil (AFMU) and 1-methylxanthine (1×) is calculated. The ratio is then logarithmically transformed and plotted in a histogram. Values greater than −0.30 are considered rapid acetylators and values less than −0.30 are considered slow acetylators. Others investigators have suggested alternative drug probes including sulfamethazine (Miesel et al. Pharmacogenetics, 7:241-246 (1997)) and isoniazid (Deguchi et al., J. Biol. Chem., 265: 12757-12760 (1990)) to accomplish the same means.

Prognosis Protocol

The invention also relates to a method of creating a prognosis protocol for a patient receiving a therapeutic metabolized by NAT-2 such as amonafide, isoniazid, phenylzine, hydrazine, dapsone, procainamide, sulfamethazine and other sulfonamides. The method includes: a) identifying patients receiving one of these drugs, b) determining whether they are rapid acetylator or a slow acetylator; and c) converting the data obtained from step (b) into a prognosis protocol. The prognosis protocol may include prediction of drug efficacy, prediction of patient outcome, prediction of drug interaction, and prediction of adverse effects. One skilled in the art can combine the nucleic acid sequence provided herein and using methods described above in determining the phenotype could develop a prognosis protocol specific for that individual. For example, studies have shown the chemotherapeutic amonifide is more toxic in rapid aceylators than in other patients. Therefore, identifying these patients using the nucleic sequence provided herein would aid the physician in designing a drug regimen which balances efficacy and toxicity.

In another example relating to the prognosis protocol, patients who are identified as slow acetylators are at risk of cutaneous hypersensitivity when administered the therapeutic trimethoprim-sulphamethoxazole (TMP-SMZ). Therefore, before prescribing a particular drug such as trimethoprim-sulphamethoxazole (TMP-SMZ), a physician could determine the patient's phentoype and if the patient is identified as a slow acetylator the physician may then prescribe an alternate therapeutic.

Clinical Trials

This invention also relates to a method to assist in the development of therapeutics through clinical trials. The method includes: a) administering a therapeutic to an individual and measuring its efficacy; b) determining by the individual's genotype and the SNPs provided herein whether the individual is a rapid acetylator and slow acetylator; and c) determining from steps (a) and (b) which therapeutic will be the most effective for that particular genotype and which will have the least adverse effects. Clinical trials typically rely on information provided by patient including age, sex, and family background. The invention provides nucleic sequences for NAT-2 which can be added to a library of SNPs and used as a identification factor of the patient in the clinical trial. As described herein, an individual's genotype can be determined by DNA sequencing methods described in Example 1.

After administering the drug, the patient's genotype can then be compared to the efficacy of the drug and any adverse effects. Based on this information, drugs can be developed specific to the genotype of the individuals which show the highest efficacy. Genotypes of patients that do not respond to the drug can be grouped together and drugs can be developed which use alternate pathways other than acetylation.

Frequency Data

The invention also relates to frequency of SNPs in various ethnic groups. This data is provided in Tables 3 and 4. The data provided in this invention reveals that five of the newly discovered SNPs, at positions −255, 51, 70, 403 and 609 (Table 3) occur exclusively in the African American sample population. Also, the SNP at position 838 occurs in African Americans and Hispanics, but not Caucasians. These SNPs can be important in predicting phenotype for these populations. The presence of these apparently population specific SNPs also demonstrate the potential for their use in differentiating between ethnic groups more accurately. Other researchers of NAT-2 have pointed out the danger in relying on ethnicity as a means of predicting likelihood of a given phenotype, as many populations with the same designation (e.g. Caucasian) may, in fact, have very different SNP/allelic frequencies (Cascorbi, I. et al., Pharmacogenetics, 9:123-127 (1999).

Forensics

The invention also relates to identifying individuals using the nucleic acid sequences provided herein. The compilation of polymorphic sites in an individual distinguishes that individual from others in a population. See generally National Research Council, The Evaluation of Forensic DNA Evidence (Eds. Pollard et al., National Academy Press, DC, 1996). These polymorphisms provide a unique set of markers which can be useful for forensic analysis. For example, one can determine whether a blood sample collected from a crime scene matches blood sample from a suspect by determining if the polymorphisms are the same in both samples. One can perform statistical analysis to determine the probability that a match of suspect and crime scene sample would occur by chance. Furthermore, Tables 3 and 4 provide the frequency of the specific polymorphisms of NAT-2 which could be used for this analysis. For further teaching see U.S. Pat. No. 5,856,1904 and WO 95/12607.

Paternity Testing

Similar to the forensic analysis above, paternity testing in determining whether a male is the father of a child could also be accomplished by the use of the nucleic acid sequence provided herein. Polymorphic sites as described above can be used in distinguishing individuals. The probability of parentage exclusion represents the probability that a random male will have a polymorphic form at a given polymorphic site makes him incompatible as the father. These statistical analyses are taught in WO 95/12607.

Diagnostic Applications

As discussed herein, NAT-2 has been associated with a variety of diseases and disorders including bladder cancer, colon cancer, prostate cancer, urothelial transitional cell carcinoma, Gilbert's disease, and leprosy. More particularly, identifying individuals who may be more susceptible to metabolizing compounds which have mutagenic-carcinogenic potential including, 2-aminofluorene, 4-aminobiphenyl, benzidine, beta-naphthylamine, and certain heterocyclic arylamines present in protein pyrolysates can be beneficial to the individual in avoiding such compunds. The inventors provide nucleic acids and SNPs which can be useful in diagnosing individuals with NAT-2 polymporphisms which are associated with these disease and affect the metabolism of the compounds described above.

Antibody-based diagnostic methods: The invention provides methods for detecting disease-associated antigenic components in a biological sample, which methods comprise the steps of: (i) contacting a sample suspected to contain an disease-associated antigenic component with an antibody specific for an disease-associated antigen, extracellular or intracellular, under conditions in which a stable antigen-antibody complex can form between the antibody and disease-associated antigenic components in the sample; and (ii) detecting any antigen-antibody complex formed in step (i) using any suitable means known in the art, wherein the detection of a complex indicates the presence of disease-associated antigenic components in the sample. It will be understood that assays that utilize antibodies directed against sequences previously unidentified, or previously unidentified as being disease-associated, which sequences are disclosed herein, are within the scope of the invention.

Many immunoassay formats are known in the art, and the particular format used is determined by the desired application. An immunoassay can use, for example, a monoclonal antibody directed against a single disease-associated epitope, a combination of monoclonal antibodies directed against different epitopes of a single disease-associated antigenic component, monoclonal antibodies directed towards epitopes of different disease-associated antigens, polyclonal antibodies directed towards the same disease-associated antigen, or polyclonal antibodies directed towards different disease-associated antigens. Protocols can also, for example, use solid supports, or may involve immunoprecipitation.

Typically, immunoassays use either a labeled antibody or a labeled antigenic component (e.g., that competes with the antigen in the sample for binding to the antibody). Suitable labels include without limitation enzyme-based, fluorescent, chemiluminescent, radioactive, or dye molecules. Assays that amplify the signals from the probe are also known, such as, for example, those that utilize biotin and avidin, and enzyme-labeled immunoassays, such as ELISA assays.

Kits suitable for antibody-based diagnostic applications typically include one or more of the following components:

(i) Antibodies: The antibodies may be pre-labeled; alternatively, the antibody may be unlabeled and the ingredients for labeling may be included in the kit in separate containers, or a secondary, labeled antibody is provided; and

(ii) Reaction components: The kit may also contain other suitably packaged reagents and materials needed for the particular immunoassay protocol, including solid-phase matrices, if applicable, and standards.

The kits referred to above may include instructions for conducting the test. Furthermore, in preferred embodiments, the diagnostic kits are adaptable to high-throughput and/or automated operation.

Nucleic-acid-based diagnostic methods: The invention provides methods for detecting disease-associated nucleic acids in a sample, such as in a biological sample, which methods comprise the steps of: (i) contacting a sample suspected to contain an disease-associated nucleic acid with one or more disease-associated nucleic acid probes under conditions in which hybrids can form between any of the probes and disease-associated nucleic acid in the sample; and (ii) detecting any hybrids formed in step (i) using any suitable means known in the art, wherein the detection of hybrids indicates the presence of the disease-associated nucleic acid in the sample. To detect disease-associated nucleic acids present in low levels in biological samples, it may be necessary to amplify the disease-associated sequences or the hybridization signal as part of the diagnostic assay. Techniques for amplification are known to those of skill in the art.

Disease-associated nucleic acids useful as probes in diagnostic methods include oligonucleotides at least about 15 nucleotides in length, preferably at least about 20 nucleotides in length, and most preferably at least about 25-55 nucleotides in length, that hybridize specifically with one or more disease-associated nucleic acids.

A sample to be analyzed, such as, for example, a tissue sample, may be contacted directly with the nucleic acid probes. Alternatively, the sample may be treated to extract the nucleic acids contained therein. It will be understood that the particular method used to extract DNA will depend on the nature of the biological sample. The resulting nucleic acid from the sample may be subjected to gel electrophoresis or other size separation techniques, or, the nucleic acid sample may be immobilized on an appropriate solid matrix without size separation.

Kits suitable for nucleic acid-based diagnostic applications typically include the following components:

(i) Probe DNA: The probe DNA may be prelabeled; alternatively, the probe DNA may be unlabeled and the ingredients for labeling may be included in the kit in separate containers; and

(ii) Hybridization reagents: The kit may also contain other suitably packaged reagents and materials needed for the particular hybridization protocol, including solid-phase matrices, if applicable, and standards.

In cases where a disease condition is suspected to involve an alteration of the disease gene, specific oligonucleotides may be constructed and used to assess the level of disease mRNA in cell affected or other tissue affected by the disease.

For example, to test whether a person has a disease gene, polymerase chain reaction can be used. Two oligonucleotides are synthesized by standard methods or are obtained from a commercial supplier of custom-made oligonucleotides. The length and base composition are determined by standard criteria using the Oligo 4.0 primer Picking program (Wojchich Rychlik, 1992). One of the oligonucleotides is designed so that it will hybridize only to the disease gene DNA under the PCR conditions used. The other oligonucleotide is designed to hybridize a segment of genomic DNA such that amplification of DNA using these oligonucleotide primers produces a conveniently identified DNA fragment. Tissue samples may be obtained from hair follicles, whole blood, or the buccal cavity. The DNA fragment generated by this procedure is sequenced by standard techniques.

Other amplification techniques besides PCR may be used as alternatives, such as ligation-mediated PCR or techniques involving Q-beta replicase (Cahill et al, Clin. Chem., 37(9):1482-5 (1991)). Products of amplification can be detected by agarose gel electrophoresis, quantitative hybridization, or equivalent techniques for nucleic acid detection known to one skilled in the art of molecular biology (Sambrook et al, Molecular Cloning: A Laboratory Manual, Cold Spring Harbor Laboratory, Cold Spring, N.Y. (1989)). Other alterations in the disease gene may be diagnosed by the same type of amplification-detection procedures, by using oligonucleotides designed to identify those alterations

Treatment of Disorders.

The present invention provides methods of screening for drugs comprising contacting such an agent with a novel protein of this invention or fragment thereof and assaying (i) for the presence of a complex between the agent and the protein or fragment, or (ii) for the presence of a complex between the protein or fragment and a ligand, by methods well known in the art. In such competitive binding assays the novel protein or fragment is typically labeled. Free protein or fragment is separated from that present in a protein:protein complex, and the amount of free (i.e., uncomplexed) label is a measure of the binding of the agent being tested to the novel protein or its interference with protein ligand binding, respectively.

This invention also contemplates the use of competitive drug screening assays in which neutralizing antibodies capable of specifically binding the NAT-2 protein compete with a test compound for binding to the NAT-2 protein or fragments thereof. In this manner, the antibodies can be used to detect the presence of any peptide which shares one or more antigenic determinants of a NAT-2 protein.

The goal of rational drug design is to produce structural analogs of biologically active proteins of interest or of small molecules with which they interact (e.g., agonists., antagonists, inhibitors) in order to fashion drugs which are, for example, more active or stable forms of the protein, or which, e.g., enhance or interfere with the function of a protein in vivo. See, e.g., Hodgson, Bio/Technology, 9:19-21 (1991). Less often, useful information regarding the structure of a protein may be gained by modeling based on the structure of homologous proteins. An example of rational drug design is the development of HIV protease inhibitors (Erickson et al, Science, 249:527-533 (1990)). In addition, peptides (e.g., NAT-2 protein) are analyzed by an alanine scan (Wells, Methods in Enzymol., 202:390-411(1991)). In this techniqae, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced banks of peptides. Selected peptides would then act as the pharmacore.

Thus, one may design drugs which have, e.g., improved NAT-2 protein activity or stability or which act as inhibitors, agonists, antagonists, etc. of NAT-2 protein activity. By virtue of the availability of cloned NAT-2 gene sequences, sufficient amounts of the NAT-2 protein may be made available to perform such analytical studies as x-ray crystallography. In addition, the knowledge of the NAT-2 protein sequence will guide those employing computer modeling techniques in place of, or in addition to x-ray crystallography.

Cells and animals that carry the NAT-2 gene or an analog thereof can be used as model systems to study and test for substances that have potential as therapeutic agents. After a test substance is applied to the cells, the transformed phenotype of the cell is determined.

The therapeutic agents and compositions of the present invention are useful for preventing or treating respiratory disease. pharmaceutical formulations suitable for therapy comprise the active agent in conjunction with one or more biologically acceptable carriers. Suitable biologically acceptable carriers include, but are not limited to, phosphate-buffered saline, saline, deionized water, or the like. Preferred biologically acceptable carriers are physiologically or pharmaceutically acceptable carriers.

The compositions include an effective amount of active agent. Effective amounts are those quantities of the active agents of the present invention that afford prophyladic protection against a respiratory disease, or which result in amelioration or cure of an existing respiratory disease. prophylactic methods incorporate a prophylactically effective amount of an active agent or composition. A prophylactically effective amount is an amount effective to prevent disease. Treatment methods incorporate a therapeutically effective amount of an active agent or composition. A therapeutically effective amount is an amount sufficient to ameliorate or eliminate the symptoms of disease The effective amount will depend upon the agent, the severity of disease and the nature of the disease, and the particular host. The amount can be determined by experimentation known in the art, such as by establishing a matrix of dosage amounts and frequencies of dosage administration and comparing a group of experimental units or subjects to each point in the matrix. The prophylactically and/or therapeutically effective amounts can be administered in one administration or over repeated administrations. Therapeutic administration can be followed by prophylactic administration, once initial clinical symptoms of disease have been resolved.

The agents and compositions can be administered topically or systemically. Systemic administration includes both oral and parental routes. Parental routes include, without limitation, subcutaneous, intramuscular, intraperitoneal, intravenous, transdermal, and intranasal administration.

Computer Readable Medium

According to another aspect of the present invention there is provided a computer readable medium comprising at least one polynucleotide sequence of the invention stored on the medium. The computer readable medium may be used, for example, in homlogy searching, mapping, haplotyping, genotyping or pharmacogenetic analysis or any other bioinformatic analysis. The reader is referred to Biomformatics, A practical guide to the analysis of genes and proteins, Edited by A D Baxevanis & B F F Quellette, John Wiley & Sons, 1988. Any computer readable medium may be used, for example, compact disk, tape, floppy disk, hard drive or computer chips.

The polynucleotide sequences of the invention, or parts thereof, particularly those relating to and identifying the single nucleotide polymorphisms identified herein represent a valuable information source, for example, to characterize individuals in terms of haplotype and other sub-groupings, such as investigation of susceptibility to treatment with particular drugs. These approaches are most easily facilitated by storing the sequence information in a computer readable medium and then using the information in standard bioinformatics programs or to search sequence databases using state of the art searching tools such as “GCG”. Thus, the polynucleotide sequences of the invention are particularly useful as components in databases useful for sequence identity and other search analyses. As used herein, storage of the sequence information in a computer readable medium and use in sequence databases in relation to ‘polynucleotide or polynucleotide sequence of the invention’ covers any detectable chemical or physical characteristic of a polynucleotide of the invention that may be reduced to, converted into or stored in a tangible medium, such as a computer disk, preferably in a computer readable form. For example, chromatographic scan data or peak data, photographic scan or peak data, mass spectrographic data, sequence gel (or other) data.

The invention provides a computer readable medium having stored thereon one or more polynucleotide sequences of the invention. For example, a computer readable medium is provided comprising and having stored thereon a member selected from the group consisting of: a polynucleotide comprising the sequence of a polynucleotide of the invention, a polynucleotide consisting of a polynucleotide of the invention, a polynucleotide which comprises part of a polynucleotide of the invention, which part includes at least one of the polymorphisms of the invention, a set of polynucleotide sequences wherein the set includes at least one polynucleotide sequence of the invention, a data set comprising or consisting of a polynucleotide sequence of the invention or a part thereof comprising at least one of the polymorphisms identified herein.

A computer based method is also provided for performing sequence identification, said method comprising the steps of providing a polynucleotide sequence comprising a polymorphism of the invention in a computer readable medium; and comparing said polymorphism containing polynucleotide sequence to at least one other polynucleotide or polypeptide sequence to identify identity (homology), i.e. screen for the presence of a polymorphism.

Gene Therapy

In recent years, significant technological advances have been made in the area of gene therapy for both genetic and acquired diseases. (Kay et al, Proc. Natl. Acad. Sci. USA, 94:12744-12746 (1997)) Gene therapy can be defined as the deliberate transfer of DNA for therapeutic purposes. Improvement in gene transfer methods has allowed for development of gene therapy protocols for the treatment of diverse types of diseases. Gene therapy has also taken advantage of recent advances in the identification of new therapeutic genes, improvement in both viral and nonviral gene delivery systems, better understanding of gene regulation, and improvement in cell isolation and transplantation. Gene therapy would be carried out according to generally accepted methods as described by, for example, Friedman, Therapy for Genetic Diseases, Friedman, Ed., Oxford University Press, pages 105-121(1991).

Vectors for introduction of genes both for recombination and for extrachromosomal maintenance are known in the art, and any suitable vector may be used. Methods for introducing DNA into cells such as electroporation, calcium phosphate co-precipitation, and viral transduction are known in the art, and the choice of method is within the competence of one skilled in the art (Robbins, Ed., Gene Therapy Protocols, Human Press, NJ (1997)). Cells transformed with a NAT-2 gene can be used as model systems to study chromosome 11 disorders and to identify drug treatments for the treatment of such disorders.

Gene transfer systems known in the art may be useful in the practice of the gene therapy methods of the present invention. These include viral and nonviral transfer methods. A number of viruses have been used as gene transfer vectors, including polyoma, i.e., SV40(Madzak et al, J. Gen. Virol., 73:1533-1536 (1992)), adenovirus (Berkner, Curr. Top. Microbiol. Immunol., 158:39-61 (1992); Berkner et al, Bio Techniques, 6:616-629 (1988); Gorziglia et al, J. Virol, 66:4407-4412 (1992); Quantin et al, Proc. Natl. Acad. Sci. USA, 89:2581-2584 (1992); Rosenfeld et al, Cell, 68:143-155 (1992); Wilkinson et al, Nucl. Acids Res., 20:2233-2239 (1992); Stratford-Perricaudet et al, Hum. Gene Ther., 1:241-256 (1990)), vaccinia virus (Mackett et al, Biotechnology, 24:495-499 (1992)), adeno-associated virus (Muzyczka, Curr. Top. Microbiol. Immunol., 158:91-123 (1992); Ohi et al, Gene, 89:279-282 (1990)), herpes viruses including HSV and EBV (Margolskee, Curr. Top. Microbiol. Immunol., 158:67-90 (1992); Johnson et al, J. Virol., 66:2952-2965 (1992); Fink et al, Hum. Gene Ther., 3:11-19 (1992); Breakfield et al, Mol. Neurobiol., 1:337-371 (1987;) Fresse et al, Biochem. Pharmacol., 40:2189-2199 (1990)), and retroviruses of avian (Brandyopadhyay et al, Mol. Cell Biol., 4:749-754 (1984); Petropouplos et al, J. Virol., 66:3391-3397 (1992)), murine (Miller, Curr. Top. Microbiol. Immunol., 158:1-24 (1992); Miller et al, Mol. Cell Biol., 5:431-437 (1985); Sorge et al, Mol. Cell Biol., 4:1730-1737 (1984); Mann et al, J. Virol., 54:401-407 (1985)), and human origin (Page et al, J. Virol., 64:5370-5276 (1990); Buchschalcher et al, J. Virol., 66:2731-2739 (1992)). Most human gene therapy protocols have been based on disabled murine retroviruses.

Nonviral gene transfer methods known in the art include chemical techniques such as calcium phosphate coprecipitation (Graham et al, Virology, 52:456-467 (1973); Pellicer et al, Science, 209:1414-1422 (1980)), mechanical techniques, for example microinjection (Anderson et al, Proc. Natl. Acad. Sci. USA, 77:5399-5403 (1980); Gordon et al, Proc. Natl. Acad. Sci. USA, 77:7380-7384 (1980); Brinster et al, Cell, 27:223-231 (1981); Constantini et al, Nature, 294:92-94 (1981)), membrane fusion-mediated transfer via liposomes (Felgner et al, Proc. Natl. Acad. Sci. USA, 84:7413-7417 (1987); Wang et al, Biochemistry, 28:9508-9514 (1989); Kaneda et al, J. Biol. Chem., 264:12126-12129 (1989); Stewart et al, Hum. Gene Ther., 3:267-275 (1992); Nabel et al, Science, 249:1285-1288 (1990); Lim et al, Circulation, 83:2007-2011 (1992)), and direct DNA uptake and receptor-mediated DNA transfer (Wolff et al, Science, 247:1465-1468 (1990); Wu et al, BioTechniques, 11:474-485 (1991); Zenke et al, Proc. Natl. Acad. Sci. USA, 87:3655-3659 (1990); Wu et al, J. Biol. Chem., 264:16985-16987 (1989); Wolff et al, BioTechniques, 11:474-485 (1991); Wagner et al, 1990; Wagner etal, Proc. Natl. Acad. Sci. USA, 88:4255-4259 (1991); Cotten et al, Proc. Natl. Acad. Sci. USA, 87:4033-4037 (1990); Curiel et al, Proc. Natl. Acad. Sci. USA, 88:8850-8854 (1991); Curiel et al, Hum. Gene Ther., 3:147-154 (1991)).

In an approach which combines biological and physical gene transfer methods, plasmid DNA of any size is combined with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged.

Liposome/DNA complexes have been shown to be capable of mediating direct in vivo gene transfer. While in standard liposome preparations the gene transfer process is non-specific, localized in vivo uptake and expression have been reported in tumor deposits, for example, following direct in situ administration (Nabel, Hum. Gene Ther., 3:399-410 (1992)).

Transgenic Animals

This invention further relates to nonhuman transgenic animals capable of expressing an exogenous or non-naturally occurring variant NAT-2 gene. Such a transgenic animal can also have one or more endogenous genes inactivated or can, instead of expressing an exogenous variant gene, have one or more endogenous analogs inactivated. Any nonhuman animal can be used; however typical animals are rodents, such as mice, rats, or guinea pigs.

Animals for testing therapeutic agents can be selected after treatment of germline cells or zygotes. Thus, expression of an exogenous NAT-2 gene or a variant can be achieved by operably linking the gene to a promoter and optionally an enhancer, and then microinjecting the construct into a zygote. See, e.g., Hogan, et al., Manipulating the Mouse Embryo, A Laboratory Manual, Cold Spring Harbor Laboratory, Cold Spring Harbor, N.Y. Such treatments include insertion of the exogenous gene and disrupted homologous genes. Alternatively, the gene(s) of the animals may be disrupted by insertion or deletion mutation of other genetic alterations using conventional techniques, such as those described by, for example, Capecchi, Science, 244:1288 (1989); Valancuis et al, Mol. Cell Biol., 11:1402 (1991); Hasty et al, Nature, 350:243 (1991); Shinkai et al, Cell, 68:855 (1992); Mombaerts et al, Cell, 68:869 (1992); Philpott et al, Science, 256:1448 (1992); Snouwaert et al, Science, 257:1083 (1992); Donehower et al, Nature, 356:215 (1992). After test substances have been administered to the animals, modulation of the disorder must be assessed. If the test substance reduces the incidence of the disorder, then the test substance is a candidate therapeutic agent. These animal models provide an extremely important vehicle for potential therapeutic products.

### EXAMPLE 1

Blood samples were collected from 88 individuals for NAT-2 genotyping. Blood samples from individuals were collected by the Interstate Blood Bank (Memphis, Tenn.) Incorporated for three ethnic groups (African Americans, Caucasians, and Hispanics) in three different geographical locations (Killen, Tex.; Memphis, Tenn.; and Miami, Fla.). Genomic DNA was isolated from these samples using an ABI model 340A automated DNA extractor (ABI, Palo Alto, Calif.).

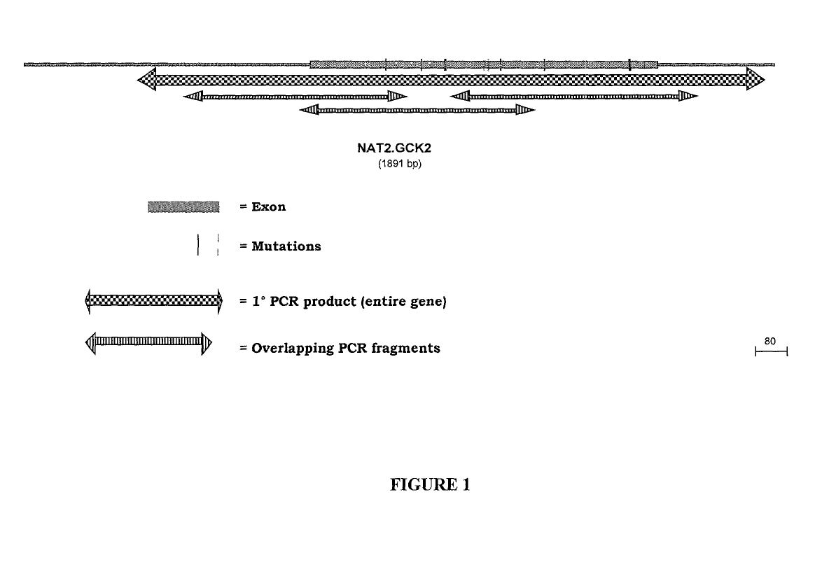

DNA templates for sequencing were generated by primary polymerase chain reaction (PCR) amplification of the entire gene for NAT-2, followed by secondary PCR amplification of three smaller overlapping fragments using chimeric primers (FIG. 1). The conditions for the PCR reaction were as follows: 25 ng of genomic DNA, 500 μM of each primary primer (Table 1), 300 nM dNTPs, 1× Boehringer-Mannheim Expand™ Long PCR Buffer 1 (Indianapolis, Ind.) and 1 unit Boehringer-Mannheim Expand™ Long PCR polymerase (Indianapolis, Ind.) were used in a final volume of 25 μl for each sample. Amplification was carried out under the following cycling conditions: initial denaturation of 94° C. for 2 minutes, followed by 32 cycles of 94° C. for 10 seconds, 50° C. for 30 seconds and 68° C. for 1.25 minutes. A final elongation step of 68° for 7 minutes was carried out followed by storage at 4° C. The primary PCR reaction was then diluted 100× with sterile water and 5 μl used in nested PCR reactions under the same conditions as described above, with the following substitutions: 350 nM dNTP and 35 cycles of 94° C. for 10 seconds, 54° C. for 30 seconds and 68° C. for 30 seconds. Ten percent of the product was examined on an agarose gel. The appropriate samples were diluted 1:25 with deionized water before sequencing.

TABLE 1
Sequences for Oligonucleotide Primer pairs

Primers for ampification of entire NAT-2 gene:

| | |
|---|---|
| 1° Forward Primer: | 5′- GTA CAG CTA AAT GGG AAA TCA AGT -3′ |
| 1° Reverse Primer: | 5′- ATG TTT TCT AGC ATG AAT CAC TCT -3′ |
| NAT2 N1P: | 5′- TGT AAA ACG ACG GCC AGT TCA TCA CCA AGA ACA CCA CAA -3′ |
| NAT2 N1R: | 5′- AGG AAA CAG CTA TGA CCA TGG TCA GAG CCC AGT ACA GAA G -3′ |
| NAT2 N2F: | 5′- TGT AAA ACG ACG GCC AGT TTT TGT TTT TCT TGC TTA GG -3′ |
| NAT2 N2R: | 5′- AGG AAA CAG CTA TGA CCA TTT TTT GGT GTT TCT TCT TTG -3′ |
| NAT2 N3F: | 5′- TGT AAA ACG ACG GCC AGT CAT TGT CGA TGC TGG GT -3′ |
| NAT2 N3R: | 5′- AGG AAA CAG CTA TGA CCA TTC TTC AAA ATA ACG TGA GGG -3′ |

M13 Chimeric PCR primers for amplification of CYP 2D6 Overlapping fragments:
(M13-21F or M13-28R portions are underlined)

Each PCR product was sequenced using DYEnamic Energy Transfer Primer Kits (AmershamPharmacia Biotech, Piscataway, N.J.). Briefly, all reactions were performed in 96 well trays. Four separate reactions, one each for A, C, G, and T, were performed for each template. Each reaction included 2μl of the sequencing reaction mix and 3 μl of diluted template. The plates were then heat sealed with foil tape and placed in a thermal cycler and cycled according to the manufacturer's recommendation. After cycling the four reactions (A, C, G and T) were pooled. 3 μl of the pooled product was transferred to a new 96 well plate and 1 μl of the manufacturer's loading dye was added to each well. 1 μl of pooled material was directly loaded onto a 48 lane gel running on an ABI 377 DNA sequencer (Palo Alto, Calif.) for 10 hour at 2.4 kV.

The analysis of the sequencing gel followed. The computer program, Polyphred (University of Washington, Seattle, Wash.) was used to assemble sequence sets for viewing with Consed (University of Washington, Seattle, Wash.), another computer program. All sequences for each study subject were assembled in a unique directory along with a monochromosomal sequence set and a color annotated reference sequence. Polyphred indicates potential polymorphic sites with purple and red tags. Two independent readers were used to examine each sequence set and assessed the validity of each tagged site.

FIGS. 2A-2B depict the wild type NAT-2 gene described by Blum et al. (DNA and Cell Bio., 9:192-203 (1990)). This figure contains the nucleotide sequence of the wild type and the amino acid sequence including the “ATG” start site (boxed). The base positions of the seven SNP's discovered are underlined in the figure. In addition, the amino acid changes are underlined.

Table 2 below contains a list the single nucleotide polymorphisms discovered. The nucleotide position according to FIGS. 2A-2B is listed in the first column. The second column describes the base change from the wild type gene. The amino acid position affected by the nucleotide base change is listed in the third column. Two single nucleotide polymorphisms at nucleotide positions, −255 and −234, were discovered in the 5′ end (untranslated region) before the start codon; therefore, these two SNP's do not encode an amino acid change. The amino acid changes of the five additional SNP's (base 51, 70, 403, 609, and 838) are listed in fourth column.

TABLE 2
SNP Positions of NAT-2

| Nucleotide | Nucleotide | | |
|---|---|---|---|
| Position | Change | Amino Acid Position | Amino Acid Change |
| −255 | C to G | 5′ untranslated region | 5′ untranslated region |
| −234 | C to T | 5′ untranslated region | 5′ untranslated region |
| 51 | C to G | 17 | N to K |
| 70 | T to A | 24 | L to I |
| 403 | C to G | 135 | L to V |
| 609 | G to T | 203 | E to D |
| 838 | G to A | 280 | V to M |

Significance of Novel SNPs

The two nucleotide substitutions at positions −255 (C to G) and −234 (C to T) are located in the untranslated region (5′ UTR) of the gene. Any changes in those regions could impact gene expression in general by altering consensus binding sites for transcriptional factors. For example, the −234 SNP apparently lies in a consensus binding sequence, gacGGAAgat (capital letters are the core sequence and the polymorphism site is identified as bold and underlined) for nuclear respiratory factor 2 (NRF-2). (Quandt, K. et al., Nucleic Acids Research, 23, 4878-4884 (1995)); Virbasius J. et al., Genes Dev, Mar, 7(3):380-92(1993)).

The invention provides further five polymorphisms of NAT-2 which alter the amino acid sequence the protein.

1. The subtitution at base 51 (C→G) results in an amino acid change at base 17 (N→K: asparagine to lysine). However, the substitution of lysine for asparagine introduces a much longer aliphatic and bulkier side chain as well as an additional positive charge at that position. Thus, it is very likely that this substitution might affect protein structure, stability, flexibility and folding behavior. A similar effect on protein stability has already been demonstrated for the previously identified mutations at positions 191 and 857 (Hein, D. W. et al. Hum. Mol. Genet., 3(5): 729-34. 1994)

2. The substitution at base 70 (T→A) in the coding sequence results in an amino acid change at base 24 (L→I: leucine to iso-leucine). Although this amino acid substitution is considered a conservative exchange (exchange by an amino acid with a similar aliphatic side chain), the amino acid is part of the consensus sequence for a Casein kinase II (ckII), a Serine Threonine kinase Consensus=(S, T)×2(D, E) identified using PROSITE). S or T is the phosphorylation site. Thus, the exchange, by altering local structure even slightly, might affect a possible phosphorylation at this site.

3. The substitution at base 403 (C→G) in the coding sequence will result in an amino acid change at base 135 (L→V: leucine to valine). This is considered a conservative substitution (aliphatic against aliphatic). However, as valine has a shorter side chain as compared to leucine, this exchange can affect the protein structure if located in a critical region. The substitution lies within close proximity to the domain identified as regions of the protein involved in substrate or cofactor binding (=Amp binding domain as identified using PROSITE). The region of amino acid residues 111 to 210 were identified as critical for protein activity (Dupret et al., 1994).

4. The substitution at base 609 (G→T) will change the amino acid at position 203 from (E→D: glutamic acid to aspartic acid). This change is conservative (exchange of an acidic residue against another). The side chain of aspartic acid is shorter than that of glutamic acid. As this exchange lies still within the region identified to be crucial for enzymatic activity in the protein, the impact on structure, activity, folding and stability can be significant.

5. The substitution at base 838 (G→A) will change amino acid 280 (V→M: valine to methionine). This change will introduce a longer aliphatic side chain with a large sulfur atom. This location of the amino acid near the C-terminus can affect structure, activity, folding and stability of the protein. The importance of the C-terminus for activity of NAT-2 is known, as glycine at 286 is also involved in substrate binding as demonstrated by a lower Km in the variant with a substitution (Hickman et al., 1995).

The combination of any or all newly discovered amino acid substitutions with those substitutions that have already been reported (see Table 4) could have additional affects on protein structure, activity, folding and stability. There is evidence to suggest that all the SNPs in NAT-2 work in concert to confer a metabolic phenotype. Using mulitiple linear regression analysis researchers were able to formulate a mathematical formula that would allow for the prediction of NAT-2 metabolic capacity based on genotype (Meisel et al., Pharmacogenomics, (1997). Their analysis indicated that all nucleic acid substitutions, even those that did not result in amino acid substitutions affected phenotype to some degree. Although they could predict phenotype in most individuals if they looked at only a few SNPs, the accuracy of the model improved when all known SNPs were taken into account. Yet they still had one individual for whom the model failed to accurately predict phenotype, suggesting that additional influential SNPs may have been present which their assay did not detect.

Frequency Data

Table 3 lists the results relating to the seven novel polymorphic sites. The first column lists ethnicity of the individuals including the number of individuals from each group. The second column details whether the individual was heterozygous or homozygous for the polymorphism listed in third though tenth columns. Frequency in the second column is the number of alleles with the polymorphism divided by the total number of alleles in the sampling. The base change and base position refers to the coordinates from FIGS. 2A-2B.

TABLE 3
SNP Frequencies for NAT-2

| | Base Change: | C to G | C to T | C to G | T to A | C to G | G to T | G to A |
|---|---|---|---|---|---|---|---|---|
| Ethnicity | Base Position: | −255 | −234 | 51 | 70 | 403 | 609 | 838 |
| All Individuals | Total Heterzyg.: | 4 | 39 | 1 | 1 | 1 | 1 | 3 |
| (88) | Total Homozyg.: | 1 | 15 | 0 | 0 | 0 | 0 | 0 |
| | Frequency: | 0.03 | 0.39 | 0.01 | 0.01 | 0.01 | 0.01 | 0.02 |
| Black American | Total Heterzyg.: | 4 | 10 | 1 | 1 | 1 | 1 | 2 |
| (29) | Total Homozyg.: | 1 | 5 | 0 | 0 | 0 | 0 | 0 |
| | Frequency: | 0.10 | 0.34 | 0.02 | 0.02 | 0.02 | 0.02 | 0.03 |
| Caucasian | Total Heterzyg.: | 0 | 14 | 0 | 0 | 0 | 0 | 0 |
| (28) | Total Homozyg.: | 0 | 8 | 0 | 0 | 0 | 0 | 0 |
| | Frequency: | 0.00 | 0.54 | 0.00 | 0.00 | 0.00 | 0.00 | 0.00 |
| Hispanic | Total Heterzyg.: | 0 | 15 | 0 | 0 | 0 | 0 | 1 |
| (31) | Total Homozyg.: | 0 | 2 | 0 | 0 | 0 | 0 | 0 |
| | Frequency: | 0.00 | 0.31 | 0.00 | 0.00 | 0.00 | 0.00 | 0.02 |

Table 4 lists the results relating to additional polymorphic sites. The first column lists ethnicity of the individuals including the number of individuals from each group. The second column details whether the individual was heterozygous or homozygous for the polymorphism listed in third though tenth columns. Frequency in the second column is the number of alleles with the polymorphism divided by the total number of alleles in the sampling. The base change and base position refers to the coordinates from FIGS. 2A-2B.

TABLE 4
SNP Frequencies for NAT-2

| | Base Change: | T to C | G to A | C to T | T to C | A to C | C to T | G to A | C to T | A to G | A to G | G to A |
|---|---|---|---|---|---|---|---|---|---|---|---|---|
| Ethnicity | Base Position: | 111 | 191 | 282 | 341 | 434 | 481 | 590 | 759 | 803 | 845 | 857 |
| All | Total Heterzyg.: | 0 | 0 | 32 | 35 | 0 | 39 | 24 | 0 | 41 | 1 | 9 |
| (88) | Total Homozyg.: | 0 | 0 | 0 | 13 | 0 | 11 | 8 | 0 | 18 | 0 | 0 |
| | Frequency: | 0.00 | 0.00 | 0.18 | 0.35 | 0.00 | 0.35 | 0.23 | 0.00 | 0.44 | 0.01 | 0.05 |
| Black American | Total Heterzyg.: | 0 | 0 | 12 | 9 | 0 | 9 | 4 | 0 | 11 | 1 | 3 |
| (29) | Total Homozyg.: | 0 | 0 | 0 | 3 | 0 | 3 | 6 | 0 | 7 | 0 | 0 |
| | Frequency: | 0.00 | 0.00 | 0.21 | 0.26 | 0.00 | 0.26 | 0.28 | 0.00 | 0.43 | 0.02 | 0.05 |
| Caucasian | Total Heterzyg.: | 0 | 0 | 7 | 12 | 0 | 15 | 8 | 0 | 13 | 0 | 0 |
| (28) | Total Homozyg.: | 0 | 0 | 0 | 8 | 0 | 6 | 2 | 0 | 9 | 0 | 0 |
| | Frequency: | 0.00 | 0.00 | 0.13 | 0.50 | 0.00 | 0.48 | 0.21 | 0.00 | 0.55 | 0.00 | 0.00 |
| Hispanic | Total Heterzyg.: | 0 | 0 | 13 | 14 | 0 | 15 | 12 | 0 | 17 | 0 | 6 |
| (31) | Total Homozyg.: | 0 | 0 | 0 | 2 | 0 | 2 | 0 | 0 | 2 | 0 | 0 |
| | Frequency: | 0.00 | 0.00 | 0.21 | 0.29 | 0.00 | 0.31 | 0.19 | 0.00 | 0.34 | 0.00 | 0.10 |

Equivalents

Those skilled in the art will recognize, or be able to ascertain using no more than routine experimentation, many equivalents to the specific embodiments of the invention described herein. Such equivalents are intended to be encompassed by the following claims.

1. An isolated nucleic acid comprising at least 15 consecutive nucleotide bases including a polymorphic site selected from the group consisting of:
a.) a C→G substitution at nucleotide −255 of SEQ ID NO:1;
b.) a C→T substitution at nucleotide −234 of SEQ ID NO:1;
c.) a C→G substitution at nucleotide 51 of SEQ ID NO:1;
d.) a T→A substitution at nucleotide 70 of SEQ ID NO:1;
e.) a C→G substitution at nucleotide 403 of SEQ ID NO:1;
f.) a G→T substitution at nucleotide 609 of SEQ ID NO:1; and
g.) a G→A substitution at nucleotide 838 of SEQ ID NO:1.

2. An isolated nucleic acid according to claim 1 comprising DNA.

3. An isolated nucleic acid according to claim 1 comprising RNA.

4. An expression vector containing the nucleic acid of claim 1.

5. A host cell containing the vector of claim 4.

6. The host cell of claim 5 which is a eukaryotic cell.

7. The host cell of claim 6 which is a human cell.

8. The host cell of claim 5 which is a prokaryotic cell.

9. An isolated allele specific primer capable of detecting a polymorphic site of SEQ ID NO:1 of claim 1.

10. An isolated allele specific oligonucleotide probe capable of detecting a polymorphic site of SEQ ID NO:1 of claim 1.

11. A diagnostic kit comprising an allele specific primer of claim 9 or allele specific oligonucleotide of claim 10.

12. An isolated nucleic acid comprising at least 50 consecutive nucleic acids of SEQ ID NO:1 containing at least one of the polymorphic sites selected from the group consisting of:
a.) a C→G substitution at nucleotide −255 of SEQ ID NO:1;
b.) a C→T substitution at nucleotide −234 of SEQ ID NO:1;
c.) a C→G substitution at nucleotide 51 of SEQ ID NO:1;
d.) a T→A substitution at nucleotide 70 of SEQ ID NO:1;
e.) a C→G substitution at nucleotide 403 of SEQ ID NO:1;
f.) a G→T substitution at nucleotide 609 of SEQ ID NO:1; and
g.) a G→A substitution at nucleotide 838 of SEQ ID NO:1.

13. An isolated nucleic acid which hybridizes to the nucleic acid according to claim 12 under high stringency conditions.

14. An expression vector containing the nucleic acid according to claim 12.

15. A host cell containing the vector of claim 14

16. The host cell of claim 15 which is a eukaryotic cell.

17. The host cell of claim 16 which is a human cell.

18. The host cell of claim 15 which is a prokaryotic cell.

19. An isolated polypeptide comprising at least 5 consecutive amino acid bases, one or more of which are encoded by the nucleotides at a polymorphic site of claim 1 or its complement.

20. An isolated polypeptide comprising at least 5 consecutive amino acid bases including a polymorphic site selected from the group consisting of:
a.) a N→K substitution at amino acid position 17 of SEQ ID NO:2;
b.) a L→I substitution at amino acid position 24 of SEQ ID NO:2;
c.) a L→V substitution at amino acid position 135 of SEQ ID NO:2;
d.) a E→D substitution at amino acid position 203 of SEQ ID NO:2; and
e.) a V→M substitution at amino acid position 280 of SEQ ID NO:2.

21. An isolated amino acid sequence having 80% identity to the amino acid sequence according to claim 20.

22. An antibody or antibody fragment which binds to an amino acid sequence of claim 19.

23. An antibody or antibody fragment which binds to an amino acid sequence of claim 20.

24. An antibody or antibody fragment which binds to an amino acid sequence of claim 21.

25. An antisense oligonucleotide comprising at least 5 nucleotide bases of a polymorphic site claim 1.

26. A method of detecting a nucleic acids of claim 1 comprising a method selected from the group consisting of: restriction-fragment-length-polymorphism detection based on allele-specific restriction-endonuclease cleavage, hybridization with allele-specific oligonucleotide probes, oligonucleotide arrays, allele-specific PCR, mismatch-repair detection (MRD), denaturing-gradient gel electrophoresis (DGGE), single-strand-conformation-polymorphism detection (SSCP), RNAase cleavage at mismatched base-pairs, chemical or cleavage of heteroduplex DNA, methods based on allele specific primer extension, genetic bit analysis (GBA), the oligonucleotide-ligation assay (OLA), the allele-specific ligation chain reaction (LCR), gap, radioactive and/or fluorescent DNA sequencing, and peptide nucleic acid (PNA) assays.

27. A method of identifying a polymorphism of SEQ ID NO:1 in a mammal, comprising the steps of:
a.) preparing a sample of cells or tissue of the mammal;
b.) probing the tissue or cell with all or a portion of a polymorphism of SEQ ID NO:1 of claim 1 under conditions wherein hybridized DNA can be produced;
c.) identifying the hybridized DNA; and
d.) cloning and sequencing the hybridized DNA to obtain and identify the NAT-2 gene in the mammal.

28. A method of treating a NAT-2 disorder comprising administering a molecule which binds to an endogenous analog of NAT-2.

29. A method of treating a NAT-2 disorder comprising administering a compound which is an agonist or an antagonist of the nucleic acid sequence of claim 1, or a variant or fragment thereof.

30. The method of claim 28 wherein the antagonist is an antibody or an antibody fragment.

31. A method of labeling an individual in a clinical trial comprising:
a.) producing a library of SNPs including the polymorphic sites of SEQ ID NO:1 of claim 1 and their respective phenotype;
b.) sequencing an individuals NAT-2 gene;
c.) matching the genotype from (b) with the phenotype in (a).

32. A method of creating a prognosis protocol comprising identifying patients receiving at least one NAT-2 drug,
a.) determining whether they are rapid acetylator or a slow acetylator; and
b.) converting the data obtained from step (b) into a prognosis protocol.

33. A method of identifying therapeutic compositions which are efficacious in individuals comprising:
a) administering a therapeutic composition to an individual and measuring its efficacy;
b) determining by the individual's genotype and the polymorphic sites of SEQ ID NO:1 of claim 1 whether the individual is a rapid acetylator and slow acetylator;
c) determining from steps (a) and (b) which therapeutic composition will be the most effective for that particular genotype and which will have the least adverse effects.

34. A method of identifying an individual comprising:
a.) sequencing an individual's NAT-2 gene;
b.) comparing the results in (a) to the frequency of NAT-2 in the population as listed in Table 3;
c.) using the data from (b) with other polymorphic sites in the human genome to statistically conclude the likelihood of the set of SNPs from this individual as compared to the general population.

35. A method of genetically linking a first individual to a second individual comprising:
a.) sequencing the NAT-2 gene of the first individual;
b.) sequencing the NAT-2 genes of the parents of the second individual;
c.) comparing the particular SNPs from the two parents with the SNPs of the second individual;
d.) matching SNPs of the parents of the second individual and assessing, through statistical means utilizing the frequency in Table 3, the likelihood of this frequency of SNPs in the general population.

36. A computer readable medium comprising at least one nucleic acid of claim 1.
